(19) United States
(12) Patent Application Publication
(10) Pub. No.: US 2007/0274940 A1
(43) Pub. Date: Nov. 29, 2007

(54) Method of enhancing the control of viruses on skin

(75) Inventors: Janice L. Fuls, Fountain Hills, AZ (US); Nancy D. Rodgers, Chandler, AZ (US); Priscilla S. Fox, Phoenix, AZ (US); James Dalton, Scottsdale, AZ (US); Harry E. Towner, Scottsdale, AZ (US); Daniel E. Pedersen, Cottage Grove, MN (US); John J. Rolando, Woodbury, MN (US); Richard K. Staub, Lakeville, MN (US)

Correspondence Address: MARSHALL, GERSTEIN & BORUN LLP (DIAL), 233 S. WACKER DRIVE, 6300 SEARS TOWER, CHICAGO, IL, 60606, US

(73) Assignee: THE DIAL CORPORATION, Scottsdale, AZ (US)

(21) Appl. No.: 11/805,392

(22) Filed: May 23, 2007

Related U.S. Application Data
(60) Provisional application No. 60/808,428, filed on May 25, 2006.
(60) Provisional application No. 60/811,354, filed on Jun. 6, 2006.

(51) Int. Cl. A61K 31/185; A61K 31/19; A61K 31/045

(52) U.S. Cl. 424/70.1; 514/553; 514/557; 514/724

(57) ABSTRACT

A method of imparting a rapid antiviral effectiveness, and a persistent antiviral effectiveness, to an animate surface is disclosed. The method includes first treating the animate surface with a preconditioning composition, followed by treating the animate surface with an antiviral composition.

### CROSS-REFERENCE TO RELATED APPLICATIONS

This application claims the benefit of U.S. Provisional Patent Application No. 60/808,428, filed May 25, 2006 and U.S. Provisional Patent Application No. 60/811,354, filed Jun. 6, 2006.

### FIELD OF THE INVENTION

The present invention relates to a method of imparting a rapid and persistent viral control to animate surfaces. More particularly, the present invention relates to a method of treating skin with a preconditioning cleansing composition, for example, an antibacterial or a neutral to mildly acidic cleansing composition, followed by treating the skin with an antiviral composition comprising an organic acid. The two-step process first preconditions the skin by removing soil and sebum and by standardizing the skin pH, preferably at a slightly acidic value, followed by use of the antiviral composition to inactivate or destroy viruses, such as rhinoviruses. The use of a preconditioning composition and an antiviral composition provide a substantial reduction in viral populations within one minute of contact with the antiviral composition and imparts a persistent antiviral activity to the skin. In some embodiments, the antiviral composition provides a barrier layer, or film, of the organic acid on treated skin to impart a persistent antiviral activity to the skin.

### BACKGROUND OF THE INVENTION

Human health is impacted by a variety of microbes encountered on a daily basis. In particular, contact with various microbes in the environment can lead to an illness, possibly severe, in mammals. For example, microbial contamination can lead to a variety of illnesses, including, but not limited to, food poisoning, a streptococcal infection, anthrax (cutaneous), athlete's foot, cold sores, conjunctivitis (“pink eye”), coxsackievirus (hand-foot-mouth disease), croup, diphtheria (cutaneous), ebolic hemorrhagic fever, and impetigo.

It is known that washing body parts (e.g., hand washing) and hard surfaces (e.g., countertops and sinks) can significantly decrease the population of microorganisms, including pathogens. Therefore, cleaning skin and other animate and inanimate surfaces to reduce microbial populations is a first defense in removing such pathogens from these surfaces, and thereby minimizing the risk of infection.

Viruses are a category of pathogens of primary concern. Viral infections are among the greatest causes of human morbidity, with an estimated 60% or more of all episodes of human illness in developed countries resulting from a viral infection. In addition, viruses infect virtually every organism in nature, with high virus infection rates occurring among all mammals, including humans, pets, livestock, and zoo specimens.

Viruses exhibit an extensive diversity in structure and life cycle. A detailed description of virus families, their structures, life cycles, and modes of viral infection is discussed in Fundamental Virology, 4th Ed., Eds. Knipe & Howley, Lippincott Williams & Wilkins, Philadelphia, Pa., 2001.

Simply stated, virus particles are intrinsic obligate parasites, and have evolved to transfer genetic material between cells and encode sufficient information to ensure their propagation. In a most basic form, a virus consists of a small segment of nucleic acid encased in a simple protein shell. The broadest distinction between viruses is the enveloped and nonenveloped viruses, i.e., those that do or do not contain, respectively, a lipid-bilayer membrane.

Viruses propagate only within living cells. The principal obstacle encountered by a virus is gaining entry into the cell, which is protected by a cell membrane of thickness comparable to the size of the virus. In order to penetrate a cell, a virus first must become attached to the cell surface. Much of the specificity of a virus for a certain type of cell lies in its ability to attach to the surface of that specific cell. Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell. Therefore, in order to control a viral infection, it is important to rapidly kill a virus that contacts the skin, and ideally to provide a persistent antiviral activity on the skin, or a hard surface, in order to control viral infections.

For example, rhinoviruses, influenza viruses, and adenoviruses are known to cause respiratory infections. Rhinoviruses are known to cause respiratory infections. Rhinoviruses are members of the picornavirus family, which is a family of “naked viruses” that lack an outer envelope. The human rhinoviruses are so termed because of their special adaptation to the nasopharyngeal region, and are the most important etiological agents of the common cold in adults and children. Officially there are 102 rhinoviruses serotypes. Most of the picornaviruses isolated from the human respiratory system are acid labile, and this lability has become a defining characteristic of rhinoviruses.

Rhinovirus infections are spread from person to person by direct contact with virus-contaminated respiratory secretions. Typically, this contact is in the form of physical contact with a contaminated surface, rather than via inhalation of airborne viral particles.

Rhinovirus can survive on environmental surfaces for hours after initial contamination, and infection is readily transmitted by finger-to-finger contact, and by contaminated environmental surface-to-finger contact, if the newly contaminated finger then is used to rub an eye or touch the nasal mucosa. Therefore, virus contamination of skin and environmental surfaces should be minimized to reduce the risk of transmitting the infection to the general population.

Several gastrointestinal infections also are caused by viruses, particularly rotaviruses. For example, Norwalk virus causes nausea, vomiting (sometimes accompanied by diarrhea), and stomach cramps. This infection typically is spread from person to person by direct contact. Acute hepatitis A viral infection similarly can be spread by direct contact between one infected person and a nonimmune individual by hand-to-hand, hand-to-mouth, or aerosol droplet transfer, or by indirect contact when an uninfected individual comes into contact with a hepatitis A virus-contaminated solid object. Norovirus causes nausea, vomiting (sometimes accompanied by diarrhea), and stomach cramps. This infection typically is spread from person to person by direct contact. Numerous other viral infections are spread similarly. The risk of transmitting such viral infections can be reduced significantly by inactivating or removing viruses from the hands and other environmental surfaces.

Common household phenol/alcohol disinfectants are effective in disinfecting contaminated environmental surfaces, but lack persistent virucidal activity. Hand washing is highly effective in disinfecting contaminated fingers, but again suffers from a lack of persistent activity. These shortcomings illustrate the need for improved virucidal methods having a persistent activity against viruses, such as rhinoviruses and rotaviruses.

Antibacterial personal care compositions are known in the art. In particular, antibacterial cleansing compositions, which typically are used to cleanse the skin and to destroy bacteria present on the skin, especially the hands, arms, and face of the user, are well-known commercial products. Antibacterial compositions are used, for example, in the health care industry, food service industry, meat processing industry, and in the private sector by individual consumers. The widespread use of antibacterial compositions indicates the importance consumers place on controlling bacteria populations on skin.

The paradigm for antibacterial compositions is to provide a substantial and broad spectrum reduction in bacterial populations quickly and without adverse side effects associated with toxicity and skin irritation. Such antibacterial compositions are disclosed in U.S. Pat. Nos. 6,107,261 and 6,136,771, each incorporated herein by reference.

One class of antibacterial personal care compositions is the hand sanitizer gels. This class of compositions is used primarily by medical personnel to disinfect the hands and fingers. A hand sanitizer gel is applied to, and rubbed into, the hands and fingers, and the composition is allowed to evaporate from the skin.

Hand sanitizer gels contain a high percentage of an alcohol, like ethanol. At the high percent of alcohol present in the gel, the alcohol itself acts as a disinfectant. In addition, the alcohol quickly evaporates to obviate wiping or rinsing skin treated with the sanitizer gel. Hand sanitizer gels containing a high percentage of an alcohol, i.e., about 40% or greater by weight of the composition, do not provide a persistent microbe control and have a tendency to dry and irritate the skin.

Hand sanitizer gels are used by consumers to effectively sanitize the hands, without, or after, washing with soap and water, by rubbing the hand sanitizer gel on the surface of the hands. Current commercial hand sanitizer gels rely on high levels of alcohol for disinfection and evaporation, and thus suffer from disadvantages. Specifically, because of the volatility of ethanol, the primary antibacterial agent does not remain on the skin after use, thus failing to provide a persistent antibacterial effect.

At alcohol concentrations below 60%, ethanol is not recognized as an antiseptic. Thus, in compositions containing less than 60% alcohol, an additional antibacterial compound is present to provide antibacterial activity. Prior disclosures, however, have not addressed the issue of which composition ingredient in such an antibacterial composition provides microbe control. Therefore, for formulations containing a reduced alcohol concentration, the selection of an antibacterial agent that provides both a rapid antibacterial effect and a persistent antibacterial benefit is difficult.

Antibacterial cleansing compositions typically contain an active antibacterial agent, a surfactant, and various other ingredients, for example, dyes, fragrances, pH adjusters, thickeners, skin conditioners, and the like, in an aqueous and/or alcoholic carrier. Several different classes of antibacterial agents have been used in antibacterial cleansing compositions. Examples of antibacterial agents include a bisguanidine (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxydiphenylether).

Antibacterial compositions based on such antibacterial agents exhibit a wide range of antibacterial activity, ranging from low to high, depending on the microorganism to be controlled and the particular antibacterial composition. Most commercial antibacterial compositions generally offer a low to moderate antibacterial activity, and no reported antiviral activity.

Antibacterial activity is assessed against a broad spectrum of microorganisms, including both Gram positive and Gram negative microorganisms, as the log reduction, or alternatively the percent reduction, in microbial populations provided by the antibacterial composition. A 1-3 log reduction is preferred, a log reduction of 3-5 is most preferred, whereas a log reduction of less than 1 is least preferred, for a particular contact time, generally ranging from 15 seconds to 5 minutes. Thus, a highly preferred antibacterial composition exhibits a 3-5 log reduction against a broad spectrum of microorganisms in a short contact time.

Virus control poses a more difficult problem than bacterial control. By sufficiently reducing bacterial populations, the risk of bacterial infection is reduced to acceptable levels. Therefore, a rapid antibacterial kill is desired. With respect to viruses, however, not only is a rapid kill desired, but a persistent antiviral activity also is required. This difference is because merely reducing a virus population is insufficient to reduce infection. In theory, a single virus can cause infection. Therefore, an essentially total, and persistent, antiviral activity is required, or at least desired, for an effective antiviral cleansing composition.

U.S. Pat. No. 6,110,908 discloses a topical antiseptic containing a C₂₋₃ alcohol, a free fatty acid, and zinc pyrithione. U.S. Pat. No. 5,776,430 discloses a topical antibacterial cleaner containing chlorhexidine and an alcohol. The compositions contain about 50% to 60%, by weight, denatured alcohol and about 0.65% to 0.85%, by weight, chlorhexidine. The composition is applied to the skin, scrubbed into the skin, then rinsed from the skin.

European Patent Application 0 604 848 discloses a gel-type hand disinfectant containing an antibacterial agent, 40% to 90% by weight of an alcohol, and a polymer and a thickening agent in a combined weight of not more than 3% by weight. The gel is rubbed into the hands and allowed to evaporate to provide disinfected hands. The disclosed compositions often do not provide immediate sanitization and do not provide persistent antibacterial efficacy.

U.S. Pat. Nos. 6,107,261 and 6,136,771 disclose highly effective antibacterial compositions containing a phenolic antibacterial agent. These patents disclose compositions that solve the problem of controlling bacteria on skin and hard surfaces, but are silent with respect to controlling viruses.

U.S. Pat. Nos. 5,968,539; 6,106,851; and 6,113,933 disclose antibacterial compositions having a pH of about 3 to about 6. The compositions contain an antibacterial agent, an anionic surfactant, and a proton donor.

Antiviral compositions disclosed as inactivating or destroying pathogenic viruses, including rhinovirus, rotavirus, influenza virus, parainfluenza virus, respiratory syncytial virus, and Norwalk virus, also are known. For example, U.S. Pat. No. 4,767,788 discloses the use of glutaric acid to inactivate or destroy viruses, including rhinovirus. U.S. Pat. No. 4,975,217 discloses compositions containing an organic acid and an anionic surfactant, for formulation as a soap or lotion, to control viruses. U.S. Patent Publication 2002/0098159 discloses the use of a proton donating agent and a surfactant, including an antibacterial surfactant, to effect antiviral and antibacterial properties.

U.S. Pat. No. 6,034,133 discloses a virucidal hand lotion containing malic acid, citric acid, and a C₁₋₆ alcohol. U.S. Pat. No. 6,294,186 discloses combinations of a benzoic acid analog, such as salicyclic acid, and selected metal salts as being effective against viruses, including rhinovirus. U.S. Pat. No. 6,436,885 discloses a combination of known antibacterial agents with 2-pyrrolidone-5-carboxylic acid, at a pH of 2 to 5.5, to provide antibacterial and antiviral properties.

Organic acids in personal washing compositions also have been disclosed. For example, WO 97/46218 and WO 96/06152 disclose the use of organic acids or salts, hydrotropes, triclosan, and hydric solvents in a surfactant base for antibacterial cleansing compositions. These publications are silent with respect to antiviral properties.

Hayden et al., Antibacterial Agents and Chemotherapy, 26:928-929 (1984), discloses interrupting the hand-to-hand transmission of rhinovirus colds through the use of a hand lotion having residual virucidal activity. The hand lotions, containing 2% glutaric acid, were more effective than a placebo in inactivating certain types of rhinovirus. However, the publication discloses that the glutaric acid-containing lotions were not effective against a wide spectrum of rhinovirus serotypes.

A virucidal tissue designed for use by persons infected with the common cold, and including citric acid, malic acid, and sodium lauryl sulfate, is known. Hayden et al., Journal of Infectious Diseases, 152:493-497 (1985), however, reported that use of paper tissues, either treated with virus-killing substances or untreated, can interrupt the hand-to-hand transmission of viruses. Hence, no distinct advantage in preventing the spread of rhinovirus colds can be attributed to the compositions incorporated into the virucidal tissues.

U.S. Pat. No. 6,805,874 discloses a method wherein the skin is pretreated with a composition that adjusts the skin pH to slightly acidic and that forms a protective lipo-regenerating layer on the skin. The skin then is treated for a specific dermatologic condition, e.g., psoriasis, and the lipo-regenerating layer from the pretreatment reduces skin irritation.

A composition that imparts a persistent activity against viruses has been difficult to achieve because the active antiviral agents in the antiviral composition must remain on skin. Furthermore, the skin may be dirty or oily which impedes adherence of the active antiviral agents to the skin and facilitates removal of the active antiviral agents from the skin.

Accordingly, a need exists for a method of treating the skin that (a) is highly efficacious against viruses in a short time period, and (b) can impart a persistent and broad spectrum antiviral activity to skin. A method of providing a heightened and extended level of viral reduction on animate surfaces is provided by the method of the present invention.

### SUMMARY OF THE INVENTION

The present invention is directed to a method of imparting a rapid and a persistent viral control to animate surfaces, and particularly human skin. More particularly, the present invention is directed to a two-step method of imparting an effective and persistent control of viruses on skin. The first step comprises treating the skin with a preconditioning composition to cleanse the skin and/or to standardize and, preferably, slightly reduce, skin pH. The second step comprises treating the preconditioned skin with an antiviral composition, wherein the antiviral composition has a pH of about 5 or less.

The present method provides a rapid and persistent control of viruses, and particularly nonenveloped viruses. In one embodiment, the preconditioning composition is an antibacterial composition. In another embodiment, the preconditioning composition is a cleansing composition, and preferably a neutral to mildly acidic cleansing composition. The preconditioning composition is applied to the skin, then typically is rinsed from the skin to remove soil and sebum, optionally control bacteria, standardize skin pH, and, preferably, render the skin slightly acidic.

After treatment with the preconditioning composition, the skin is treated with an antiviral composition to inactivate or destroy viruses harmful to human health, particularly nonenveloped viruses, like acid-labile viruses, and especially rhinoviruses, other acid-labile picornaviruses, and rotaviruses. Preferably, the antiviral composition is a leave-on composition that is not rinsed from the skin.

Therefore, one aspect of the present invention is to provide a method of imparting a substantial, broad spectrum, and persistent virus control to treated skin.

In yet another aspect of the present invention, the antiviral composition comprises:

(a) about 25% to 75%, by weight, of a disinfecting alcohol, like a C₁₋₆ alcohol;

(b) a virucidally effective amount of one or more organic acid; and

(c) an optional gelling agent;

(d) water,

wherein the composition has a pH of about 5 or less.

The antiviral composition typically comprises one or more organic acid. A present antiviral composition is free of intentionally added cleansing surfactants, such as anionic, cationic, and ampholytic surfactants. In preferred embodiments, the compositions comprise a gelling agent. The antiviral compositions also can contain an optional active antibacterial agent, such as a phenolic or a quaternary ammonium antibacterial agent.

Another aspect of the present invention is to provide a liquid, antiviral composition that provides an essentially continuous layer or film of the organic acid on treated skin to impart a persistent antiviral activity to the treated skin.

A present antiviral composition provides a rapid and persistent control of nonenveloped viruses, in addition to a fast, broad spectrum bacteria kill. The compositions also provide a persistent control of influenza viruses and noroviruses. In one embodiment, the organic acid has a water-octanol partition coefficient, expressed as log P, of less than one, and the composition exhibits a synergistic activity against nonenveloped viruses. In another embodiment, the organic acid has a log P of one or greater, and the composition exhibits a synergistic activity agent bacteria. In yet another embodiment, the organic acid comprises a first organic acid having a log P less than one and a second organic acid having a log P of one or greater, and the composition exhibits a synergistic activity against both nonenveloped viruses and bacteria.

Accordingly, one aspect of the present invention is to provide an antiviral composition that also is highly effective at killing a broad spectrum of bacteria, including Gram positive and Gram negative bacteria such as S. aureus, S. choleraesuis, E. coli, and K. pneumoniae, while simultaneously inactivating or destroying viruses harmful to human health, particularly nonenveloped viruses, like acid-labile viruses, and especially rhinoviruses, and other acid-labile picornaviruses. Influenza viruses and noroviruses also are controlled.

Another aspect of the present invention is to provide an antiviral composition having antibacterial and antiviral activity comprising (a) a disinfecting alcohol and (b) an organic acid that is substantive to the skin, and/or that fails to penetrate the skin, and/or that resists rinsing from the skin, and/or that forms an essentially continuous barrier layer on the skin, for example, hydrophobic monocarboxylic acids, polycarboxylic acids, polymeric acids having a plurality of carboxylic, phosphate, sulfonate, and/or sulfate moieties, or mixtures thereof, and (c) water, wherein the composition has a pH of about 5 or less. Such organic acids typically have a log P of less than one, and the compositions are effective against a broad spectrum of bacteria and exhibit a synergistic activity against nonenveloped viruses and noroviruses. The compositions also are effective against influenza viruses. The persistent antiviral activity is attributed, in part, to a residual layer or film of the organic acid on a treated surface, which resists removal from the skin after several rinsings, and during normal daily routines for a period of several hours.

Still another aspect of the present invention is to provide an antiviral composition comprising (a) a disinfecting alcohol and (b) an organic acid selected from the group consisting of monocarboxylic acids, polycarboxylic acids, polymeric acids having a plurality of carboxylic, phosphate, sulfonate, and/or sulfate moieties, or mixtures thereof, and (c) water, wherein the composition has a pH of about 5 or less, and the organic acid has a log P of one or greater. Preferred compositions comprise one or more polycarboxylic acid, a polymeric acid, and a gelling agent. These compositions provide an effective and persistent control of nonenveloped viruses and exhibit a synergistic activity against Gram positive and Gram negative bacteria.

Another aspect of the present invention is to provide an antiviral composition that exhibits a log reduction against nonenveloped viruses, such as acid-labile viruses, including rhinovirus serotypes, such as Rhinovirus 1a, Rhinovirus 2, Rhinovirus 14, and Rhinovirus 4, and rotavirus serotypes, such as Rotavirus Wa, of at least 4 after 30 seconds of contact. The antiviral composition also provides a log reduction against nonenveloped viruses of about 3 for at least about five hours, and at least 2 for about six hours, after application with a 30 second contact time. In some embodiments, the antiviral composition provides a log reduction of 2 against nonenveloped viruses for up to about eight hours.

Yet another aspect of the present invention is to provide a method of interrupting transmission of a virus from animate and inanimate surfaces to an animate surface, especially human skin. Especially provided is a method for controlling the transmission of nonenveloped viruses, particularly rhinoviruses, by effectively controlling viruses present on human skin and continuing to control the viruses for a period of about four or more hours, and up to about eight hours, after application of the composition to the skin.

In preferred embodiments, the composition provides an essentially continuous layer or film of the organic acid on a treated surface to impart a persistent antiviral activity to the treated surface. In other preferred embodiments, the composition is free of an intentionally-added surfactant.

The present method preferably utilizes antiviral compositions comprising one or more polycarboxylic acid, a polymeric acid, and a gelling agent. These compositions provide an effective and persistent control of viruses and exhibit a synergistic activity against Gram positive and Gram negative bacteria.

Another aspect of the present invention is to provide a method that utilizes an antiviral composition that exhibits a substantial, and preferably persistent, control of noroviruses, and has a pH of about 2 to about 5.

Yet another aspect of the present invention is to provide a method that exhibits a log reduction against Gram positive bacteria (i.e., S. aureus) of at least 2 after 30 seconds of contact.

Still another aspect of the present invention is to provide a method that exhibits a log reduction against Gram negative bacteria (i.e., E. coli) of at least 2.5 after 30 seconds of contact.

Another aspect of the present invention is to provide a method wherein an antiviral composition is applied to the skin and resists rinsing from the skin, e.g., at least 50%, at least 60%, and preferably at least 70% of the nonvolatile components of an applied composition remains on treated skin after three water rinsings and an effective antiviral amount of the composition remains on the skin after ten water rinsings.

Another aspect of the present invention is to provide consumer products based on the method of the present invention, for example, a dual dispensing system comprising the preconditioning composition and the antiviral composition in separate packaging designed for separate dispensing of the compositions. To achieve the full advantage of the present invention, the dual dispensing system measures effective doses of the preconditioning and antiviral compositions, i.e., a sufficient dose of the preconditioning composition to cleanse the skin and standardize skin pH, and a sufficient dose of the antiviral composition to impart a persistent antiviral efficacy to the treated skin.

The preconditioning composition and antiviral composition, individually, can be a rinse-off product or a leave-on product. Preferably, the preconditioning composition is rinsed from the skin, and the antiviral composition remains on the skin to allow the volatile components of the composition evaporate and the nonvolatile antiviral agents to remain on the skin.

A further aspect of the present invention is to provide a method of quickly controlling a wide spectrum of viruses on animal tissue, including human tissue, by contacting the tissue, like the dermis, first with a preconditioning composition, then with an antiviral composition, each for a sufficient time, for example, about 15 seconds to 5 minutes or longer, e.g., about one hour, to reduce virus populations to a desired level. A further aspect of the present invention is to provide a method that imparts a persistent control of viruses on animal tissue.

Still another aspect of the present invention is to provide a method of reducing or preventing the transmission of virus-mediated diseases and conditions caused by rhinoviruses, picornaviruses, adenoviruses, herpes viruses, respiratory syncytial viruses (RSV), coronaviruses, enteroviruses, and other nonenveloped viruses. The present method also prevents the transmission of diseases mediated by rotaviruses, noroviruses, and influenza viruses.

Yet another aspect of the present invention is to provide a method of interrupting transmission of a virus from animate and inanimate surfaces to an animate surface, especially human skin. Especially provided is a method for controlling the transmission of nonenveloped viruses, particularly rhinovirus, by effectively controlling viruses present on human skin and continuing to control the viruses for a period of about four or more hours, and up to about eight hours, after application of the antiviral composition to the skin.

These and other novel aspects and advantages of the present invention are set forth in the following, nonlimiting detailed description of the preferred embodiments.

### BRIEF DESCRIPTION OF THE FIGURES

FIGS. 1a and 1b are reflectance micrographs showing a barrier layer of nonvolatile components on a surface provided by application of an antiviral composition to the surface, and

FIGS. 1c and 1d are reflectance micrographs showing the absence of a barrier layer on a surface after application of a control composition to the surface.

### DETAILED DESCRIPTION OF THE PREFERRED EMBODIMENTS

Personal care products incorporating an active antibacterial agent have been known for many years. Since the introduction of antibacterial personal care products, many claims have been made that such products provide antibacterial properties. To be most effective, an antibacterial composition should provide a high log reduction against a broad spectrum of organisms in as short a contact time as possible. Ideally, the composition also should inactivate viruses.

As presently formulated, most commercial liquid antibacterial soap compositions provide a poor to marginal time kill efficacy, i.e., rate of killing bacteria. These compositions do not effectively control viruses.

Antibacterial hand sanitizer compositions typically do not contain a surfactant and rely upon a high concentration of an alcohol to control bacteria. The alcohols evaporate and, therefore, cannot provide a persistent bacterial control. The alcohols also can dry and irritate the skin.

Most current products especially lack efficacy against Gram negative bacteria, such as E. coli, which are of particular concern to human health. Compositions do exist, however, that have an exceptionally high broad spectrum antibacterial efficacy, as measured by a rapid kill of bacteria (i.e., time kill), which is to be distinguished from persistent kill. These products also lack a sufficient antiviral activity.

The present method provides an excellent broad spectrum antiviral efficacy and significantly improves antiviral efficacy compared to prior methods and compositions that incorporate a high percentage of an alcohol, i.e., 40% or greater, by weight. The basis of this improved efficacy is the discovery that a dual treatment of the skin, first with a preconditioning composition, then with an antiviral composition, substantially improves antiviral efficacy.

An important aspect of the present invention is to maintain a low skin pH for an extended time to provide a persistent antiviral activity. In preferred embodiments, this is achieved by forming an essentially continuous film of the nonvolatile antiviral composition components on the skin, which provides a reservoir of the organic acids to maintain a low skin pH.

The term “essentially continuous film” means that a residue of the nonvolatile components of the composition in the form of a barrier layer is present on at least 50%, at least 60%, at least 70%, or at least 80%, preferably at least 85% or at least 90%, and more preferably at least 95%, of the area of the treated surface area. An “essentially continuous” film is demonstrated in the reflectance micrographs of the figures, which are discussed hereafter. The term “essentially continuous film” as used herein is synonymous with the term “essentially continuous layer”, “barrier layer”, and “barrier film”.

The preconditioning composition is any cleansing composition that removes sebum and soil from the skin. Preferably, the preconditioning composition is a neutral to mildly acidic cleansing or antibacterial composition that removes soil and sebum from the skin and, preferably, slightly lowers skin pH. Regardless of the identity of the preconditioning composition, the skin pH is standardized, which improves the efficacy of the antiviral composition.

The antiviral composition comprises a disinfecting alcohol and an organic acid, and preferably an organic acid having a log P of less than about 1, such that the pH of the skin after application of the antiviral composition is sufficiently lowered to provide a persistent antiviral activity.

A disinfecting alcohol and an organic acid having a log P of less than one act synergistically to control nonenveloped viruses. A disinfecting alcohol and an organic acid having a log P of one or greater act synergistically to substantially improve antibacterial efficacy. A combination of a first organic acid having a log P less than one and a second organic acid having a log P of one or greater, with a disinfecting alcohol, provides a synergistic improvement in the control of nonenveloped viruses and Gram positive and Gram negative bacteria.

Although compositions containing an antibacterial agent, like triclosan, have demonstrated a rapid and effective antibacterial activity against Gram positive and Gram negative bacteria, control of viruses has been inadequate. Virus control on skin and inanimate surfaces is very important in controlling the transmission of numerous diseases.

For example, rhinoviruses are the most significant microorganisms associated with the acute respiratory illness referred to as the “common cold.” Other viruses, such as parainfluenza viruses, respiratory syncytial viruses (RSV), enteroviruses, and coronaviruses, also are known to cause symptoms of the “common cold,” but rhinoviruses are theorized to cause the greatest number of common colds. Rhinoviruses also are among the most difficult of the cold-causing viruses to control, and have an ability to survive on a hard dry surface for more than four days. In addition, most viruses are inactivated upon exposure to a 70% ethanol solution. However, rhinoviruses remain viable upon exposure to ethanol.

Because rhinoviruses are the major known cause of the common cold, it is important that a composition having antiviral activity is active against the rhinovirus. Although the molecular biology of rhinoviruses is now understood, finding effective methods for preventing colds caused by rhinoviruses, and for preventing the spread of the virus to noninfected subjects, has been fruitless.

It is known that iodine is an effective antiviral agent, and provides a persistent antirhinoviral activity on skin. In experimentally induced and natural cold transmission studies, subjects who used iodine products had significantly fewer colds than placebo users. This indicates that iodine is effective for prolonged periods at blocking the transmission of rhinoviral infections. Thus, the development of products that deliver both immediate and persistent antiviral activity would be effective in reducing the incidence of colds. Likewise, a topically applied composition that exhibits antiviral activity would be effective in preventing and/or treating diseases caused by other nonenveloped viruses, including acid-labile viruses.

A rotavirus also is a double-shelled virus that is stable in the environment. Rotavirus infection is an infection of the digestive tract, and is the most common cause of severe diarrhea among children, resulting in over 50,000 hospitalizations yearly in the U.S. alone. Rotaviral infections are particularly problematic in close communities, such as child care facilities, geriatric facilities, family homes, and children's hospitals.

The most common mode of transmitting rotavirus is person to person spread through contaminated hands, but transmission also can occur through ingestion of contaminated water or food, or through contact with contaminated surfaces. The rotavirus then enters the body through contact with the mouth.

It is known that washing hands and hard surfaces with soap and/or other cleansers does not kill rotavirus, but helps prevent its spread. An oral rotavirus vaccine has been approved for use in children in the U.S., but its use is not recommended because of a severe adverse side effect. Because no other effective way to eliminate rotavirus, or its spread, is currently available, workers in close communities, especially those catering to children, must adhere to strict hygienic practices to help curtail the spread of rotavirus. An improved method having enhanced antiviral efficacy, including a persistent antiviral efficacy, in inactivating rotaviruses would further curtail the spread of rotavirus infections.

Virucidal means capable of inactivating or destroying a virus. As used herein, the term “persistent antiviral efficacy” or “persistent antiviral activity” means leaving a residue or imparting a condition on animate (e.g., skin) or inanimate surfaces that provides significant antiviral activity for an extended time after application. In some embodiments, a “persistent antiviral efficacy” or “persistent antiviral activity” means leaving a barrier residue or film of antiviral agents, including organic acids, on animate (e.g., skin) or inanimate surfaces that provides significant antiviral activity for an extended time after application. The barrier residue or film can be continuous or essentially continuous, and resists removal from a treated surface during water rinsing.

A composition of the present invention provides a persistent antiviral efficacy, i.e., preferably a log reduction of at least 3, and more preferably a log reduction of at least log 4, against nonenveloped viruses, including acid-labile viruses, such as rhinovirus serotypes, within 30 seconds of contact with the composition. Antiviral activity is maintained for at least about 0.5 hour, preferably at least about one hour, and more preferably for at least about two hours, at least about three hours, or at least about four hours after contact with the composition. In some preferred embodiments, antiviral activity is maintained for about six to about eight hours after contact with the composition. The persistent antiviral activity is attributed, at least in part, to a reservoir of organic acids present in a barrier layer or film of the composition on treated skin. The barrier residue or film can be continuous, and resists removal from a treated surface during water rinsing. The methodology utilized to determine a persistent antiviral efficacy is discussed below.

The methods of the present invention are highly effective in providing a rapid and persistent control of viruses, and particularly nonenveloped viruses. The highly effective antiviral compositions comprise a disinfecting alcohol, a virucidally effective amount of an organic acid, an optional active antibacterial agent, and an optional gelling agent. Preferred embodiments comprise at least one of a polymeric acid and a gelling agent. Other preferred embodiments contain a polymeric acid and a gelling agent. The preconditioning composition and method step further enhance the efficacy of the antiviral composition. Thus, a method using mild and effective compositions that solve the problem of virus control is available to the public.

The present method provides an effective and persistent inactivation of nonenveloped viruses. The present method also provides a persistent antiviral effectiveness. Nonenveloped viruses include, but are not limited to, adenoviruses, caulimoviruses, papovaviruses, phycodna viruses, circoviruses, parvoviruses, bimaviruses, rotoviruses (including rotavirus gastroenteritis), astroviruses, caliciviruses (including Norwalk virus), potyviruses, and picornaviruses (including rhinovirus, polio virus, and hepatitis A virus). The present method also provides an effective inactivation of influenza viruses and noroviruses.

As illustrated in detail below, the present invention is directed to a method of imparting a persistent antiviral activity to treated skin. The method comprises first contacting the skin with a preconditioning composition, followed by contacting the skin with an antiviral composition. The compositions can be leave-on or rinse-off compositions. The preconditioning composition preferably is a rinse-off composition, and the antiviral composition preferably is a leave-on composition.

The following is a nonlimiting, detailed description of a preferred embodiment of the invention.

Preconditioning Step

The first step in the present process is to precondition the skin prior to application of an antiviral composition by contacting, or treating, the skin with a preconditioning composition that cleanses the skin, optionally reduces bacterial populations on the skin, and standardizes and/or slightly reduces skin pH. Preferably, the preconditioning composition removes soil and sebum from the skin, which permits a more efficacious application of the antiviral composition to the skin and enhances persistence of the virus control. In preferred embodiments, the preconditioning composition is neutral to mildly acidic and slightly reduces skin pH. Accordingly, the skin surface is preconditioned and standardized such that an organic acid present the antiviral composition is more available to inactivate and kill viruses.

As discussed below, the identity of the preconditioning composition is not limited, as long as the skin pH after the preconditioning step is less than about 6.5. Even if the preconditioning step increases skin pH, cleansed skin having a standardized pH below about 6.5 increases the efficacy of the later applied antiviral composition.

The preconditioning composition is applied to the skin in a sufficient amount to thoroughly and completely contact the desired area of the skin, often the hands. If so designed, the preconditioning composition is allowed to remain on the skin, i.e., is a leave-on composition that is not rinsed from the skin. Otherwise, after a sufficient contact time, e.g., about 15 seconds to about two minutes, the preconditioning composition is rinsed from the skin. Either immediately after, or shortly after, the preconditioning treatment, the skin is treated with an antiviral composition. The antiviral composition is applied to the skin sufficiently soon after the preconditioning step such that the skin does not become resoiled to an appreciable degree.

The preconditioning composition can be any composition that cleanses the skin. Accordingly, commercial liquid and bar soaps can be used as the preconditioning composition. For industrial and institutional applications, specially formulated hand cleansers for a particular end use also can be used. These commercial compositions often are based on anionic surfactants, which effectively clean the skin, but that have a relatively high pH and can irritate the skin if used repeatedly during a day. Compositions that leave a film on the hands, such as waterless hand cleansers, preferably are avoided or must be thoroughly rinsed from the hands.

The preconditioning composition preferably is a mild, and preferably a mildly acidic, cleansing composition, and can be an antibacterial composition. Antibacterial compositions are known in the art. Preferred antibacterial compositions are disclosed, for example, in U.S. Pat. Nos. 6,107,261 and 6,136,771, each incorporated herein by reference. An example of a useful commercial neutral to mildly acidic cleansing composition is Dial Complete, available from The Dial Corporation, Scottsdale, Ariz.

The antibacterial composition typically contains an active antibacterial agent, a surfactant, and various other ingredients, for example, dyes, fragrances, pH adjusters, thickeners, skin conditioners, and the like, in an aqueous and/or alcoholic carrier. Several different classes of antibacterial agents have been used in antibacterial cleansing compositions. Examples of antibacterial agents include a bisguanidine (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxy-diphenylether).

In particular, an antibacterial agent can be present in a preconditioning antibacterial composition in an amount of 0.1% to about 5%, and preferably about 0.1% to about 2%, and more preferably, about 0.3% to about 1%, by weight of the composition.

Antibacterial agents useful in the present invention are exemplified by the following classes of compounds used alone or in combination:

(1) Phenolic Antibacterial Agents

(a) 2-Hydroxydiphenyl Compounds

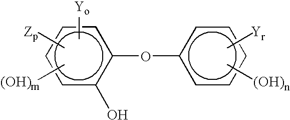

wherein Y is chlorine or bromine, Z is SO₃H, NO₂, or C₁-C₄ alkyl, r is to 3, o is 0 to 3, p is 0 or 1, m is 0 or 1, and n is 0 or 1.

In preferred embodiments, Y is chlorine or bromine, m is 0, n is 0 or 1, o is 1 or 2, r is 1 or 2, and p is 0.

In especially preferred embodiments, Y is chlorine, m is 0, n is 0, o is 1, r is 2, and p is 0.

A particularly useful 2-hydroxydiphenyl compound has a structure: 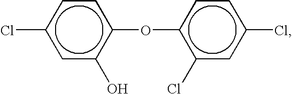
having the adopted name, triclosan, and available commercially under the tradename IRGASAN DP300, from Ciba Specialty Chemicals Corp., Greensboro, N.C. Another useful 2-hydroxydiphenyl compound is 2,2′-dihydroxy-5,5′-dibromo-diphenyl ether.

(b) Phenol Derivatives

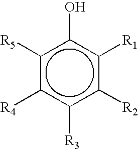

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl; and R₅ is hydro or nitro. Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsulfonic acid. Other phenol derivatives are listed in U.S. Pat. No. 6,436,885, incorporated herein by reference.

(c) Diphenyl Compounds

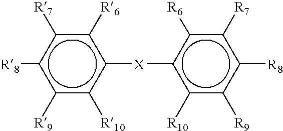

wherein X is sulfur or a methylene group, R₆ and R′₆ are hydroxy, and R₇, R′₇, R₈, R′₈, R₉, R′₉, R₁₀, and R′₁₀, independent of one another, are hydro or halo. Specific, nonlimiting examples of diphenyl compounds are hexachlorophene, tetrachlorophene, dichlorophene, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine. Other diphenyl compounds are listed in U.S. Pat. No. 6,436,885, incorporated herein by reference.

(2) Quaternary Ammonium Antibacterial Agents

Useful quaternary ammonium antibacterial agents have a general structural formula:

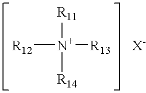

wherein at least one of R₁₁, R₁₂, R₁₃, and R₁₄ is an alkyl, aryl, or alkaryl substituent containing 6 to 26 carbon atoms. Alternatively, any two of the R substituents can be taken together, with the nitrogen atom, to form a five- or six-membered aliphatic or aromatic ring. Preferably, the entire ammonium cation portion of the antibacterial agent has a molecular weight of at least 165.

The substituents R₁₁, R₁₂, R₁₃, and R₁₄ can be straight chained or can be branched, but preferably are straight chained, and can include one or more amide, ether, or ester linkage. In particular, at least one substituent is C₆-C₂₆alkyl, C₆-C₂₋₆alkoxyaryl, C₆-C₂₆alkaryl, halogen-substituted C₆-C₂₆alkaryl, C₆-C₂₆alkylphenoxyalkyl, and the like. The remaining substituents on the quaternary nitrogen atom other than the above-mentioned substituent typically contain no more than 12 carbon atoms. In addition, the nitrogen atom of the quaternary ammonium antibacterial agent can be present in a ring system, either aliphatic, e.g., piperidinyl, or aromatic, e.g., pyridinyl. The anion X can be any salt-forming anion which renders the quaternary ammonium compound water soluble. Anions include, but are not limited to, a halide, for example, chloride, bromide, or iodide, methosulfate, and ethosulfate.

Preferred quaternary ammonium antibacterial agents have a structural formula:

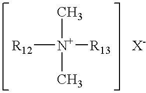

wherein R₁₂ and R₁₃, independently, are C₉-C₁₂alkyl, or R₁₂ is C₁₂-C₁₆alkyl, C₈-C₁₈alkylethoxy, or C₈-C₁₈alkylphenylethoxy, and R₁₃ is benzyl, and X is halo, methosulfate, ethosulfate, or p-toluenesulfonate. The alkyl groups R₁₂ and R₁₃ can be straight chained or branched, and preferably are linear.

The quaternary ammonium antibacterial agent in a present composition can be a single quaternary ammonium compound, or a mixture of two or more quaternary ammonium compounds. Particularly useful quaternary ammonium antibacterial agents include dialkyl(C₈-C₁₀) dimethyl ammonium chlorides (e.g., dioctyl dimethyl ammonium chloride), alkyl dimethyl benzyl ammonium chlorides (e.g., benzalkonium chloride and myristyl dimethylbenzyl ammonium chloride), alkyl methyl dodecyl benzyl ammonium chloride, methyl dodecyl xylene-bis-trimethyl ammonium chloride, benzethonium chloride, dialkyl methyl benzyl ammonium chloride, alkyl dimethyl ethyl ammonium bromide, and an alkyl tertiary amine. Polymeric quaternary ammonium compounds based on these monomeric structures also can be used in the present invention. One example of a polymeric quaternary ammonium compound is POLYQUAT®, e.g., a 2-butenyl dimethyl ammonium chloride polymer. The above quaternary ammonium compounds are available commercially under the tradenames BARDAC®, BTC®, HYAMINE®, BARQUAT®, and LONZABAC®, from suppliers such as Lonza, Inc., Fairlawn, N.J. and Stepan Co., Northfield, Ill.

Additional examples of quaternary ammonium antibacterial agents include, but are not limited to, alkyl ammonium halides, such as cetyl trimethyl ammonium bromide; alkyl aryl ammonium halides, such as octadecyl dimethyl benzyl ammonium bromide; N-alkyl pyridinium halides, such as N-cetyl pyridinium bromide; and the like. Other suitable quaternary ammonium antibacterial agents have amide, ether, or ester moieties, such as octylphenoxyethoxy ethyl dimethyl benzyl ammonium chloride, N-(laurylcocoaminoformylmethyl)pyridinium chloride, and the like. Other classes of quaternary ammonium antibacterial agents include those containing a substituted aromatic nucleus, for example, lauryloxyphenyl trimethyl ammonium chloride, cetylaminophenyl trimethyl ammonium methosulfate, dodecylphenyl trimethyl ammonium methosulfate, dodecylbenzyl trimethyl ammonium chloride, chlorinated dodecylbenzyl trimethyl ammonium chloride, and the like.

Specific quaternary ammonium antibacterial agents include, but are not limited to, behenalkonium chloride, cetalkonium chloride, cetarylalkonium bromide, cetrimonium tosylate, cetyl pyridinium chloride, lauralkonium bromide, lauralkonium chloride, lapyrium chloride, lauryl pyridinium chloride, myristalkonium chloride, olealkonium chloride, and isostearyl ethyldimonium chloride. Preferred quaternary ammonium antibacterial agents include benzalkonium chloride, benzethonium chloride, cetyl pyridinium bromide, and methylbenzethonium chloride.

(3) Anilide and Bisguanidine Antibacterial Agents

Useful anilide and bisguanadine antibacterial agents include, but are not limited to, triclocarban, carbanilide, salicylanilide, tribromosalan, tetrachlorosalicylanilide, fluorosalan, chlorhexidine gluconate, chlorhexidine hydrochloride, and mixtures thereof.

The preconditioning step does not require use of an antibacterial composition. The preconditioning step also can utilize a neutral to mildly acidic cleansing composition. Preferably, a surfactant is present in the preconditioning composition in a sufficient amount to clean the skin, but not in an amount that adversely affects the ability of an antibacterial agent to control bacteria, that provides a harsh composition, or that raises skin pH. Preferred surfactants that achieve a high level of soil and sebum removal are anionic surfactants, like a C₈-C₁₈ alkyl sulfate, a C₈-C₁₈ fatty acid salt, a C₈-C₁₈ alkyl ether sulfate having one or two moles of ethoxylation, a C₈-C₁₈ alkamine oxide, a C₈-C₁, alkyl sarcosinate, a C₈-C₁₈ sulfoacetate, a C₈-C₁₈ sulfosuccinate, a C₈-C₁₈ alkyl diphenyl oxide disulfonate, a C₈-C₁₈alkyl carbonate, a C₈-C₁₈ alpha-olefin sulfonate, a methyl ester sulfonate, and mixtures thereof. The surfactant also can be a nonionic or amphoteric surfactant to increase the mildness of the composition. Any combination of anionic, nonionic, and amphoteric surfactant also can be used in the preconditioning composition.

The preconditioning compositions, including antibacterial compositions, preferably have a pH of about 5 to about 7, and more preferably about 5 to about 6.5. The components of the preconditioning composition are not necessarily limited, and are the same type of ingredients that are present in an antibacterial composition discussed above, without the presence of an antibacterial agent. The cleansing composition typically contains surfactants, including anionic surfactants, and preferably mild nonionic and amphoteric surfactants.

The preconditioning composition, therefore, also can be a cleansing composition based on a surfactant. The surfactant is included in a preconditioning composition in an amount of 0.1% to about 15%, and typically 0.1% to about 10%, by weight, of the composition. More typically, the preconditioning composition contains 0.1% to about 7%, by weight of the surfactant. The surfactant is stable at the pH of the composition and is compatible with the other ingredients present in the composition.

The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

The compositions, therefore, can contain an anionic surfactant having a hydrophobic moiety, such as a carbon chain including about 8 to about 30 carbon atoms, and particularly about 12 to about 20 carbon atoms, and further has a hydrophilic moiety, such as sulfate, sulfonate, carbonate, phosphate, or carboxylate. Often, the hydrophobic carbon chain is etherified, such as with ethylene oxide or propylene oxide, to impart a particular physical property, such as increased water solubility or reduced surface tension to the anionic surfactant.

Suitable anionic surfactants include, but are not limited to, compounds in the classes known as alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylaryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates, acyl glutamates, alkyl sulfoacetates, acylated peptides, acyl lactylates, anionic fluoro surfactants, and mixtures thereof. Additional anionic surfactants are listed in McCutcheon's Emulsifiers and Detergents, 1993 Annuals, (hereafter McCutcheon's), McCutcheon Division, MC Publishing Co., Glen Rock, N.J., pp. 263-266, incorporated herein by reference. Numerous other anionic surfactants, and classes of anionic surfactants, are disclosed in U.S. Pat. No. 3,929,678 and U.S. Patent Publication No. 2002/0098159, each incorporated herein by reference.

Specific, nonlimiting classes of anionic surfactants useful in the present invention include, but are not limited to, a C₈-C₁₈ alkyl sulfonate, a C₈-C₁₈ alkyl sulfate, a C₈-C₁₈ fatty acid salt, a C₈-C₁₈ alkyl ether sulfate having one or two moles of ethoxylation, a C₈-C₁₈ alkamine oxide, a C₈-C₁₈ alkoyl sarcosinate, a C₈-C₁₈ sulfoacetate, a C₈-C₁₈ sulfosuccinate, a C₈-C₁₈ alkyl diphenyl oxide disulfonate, a C₈-C₁₈ alkyl carbonate, a C₈-C₁₈ alpha-olefin sulfonate, a methyl ester sulfonate, and mixtures thereof. The C₈-C₁₈ alkyl group contains eight to eighteen carbon atoms, and can be straight chain (e.g., lauryl) or branched (e.g., 2-ethylhexyl). The cation of the anionic surfactant can be an alkali metal (preferably sodium or potassium), ammonium, C₁-C₄ alkylammonium (mono-, di-, tri-), or C₁-C₃ alkanolammonium (mono-, di-, tri-). Lithium and alkaline earth cations (e.g., magnesium) can be used, but are not preferred.

Specific surfactants include, but are not limited to, lauryl sulfates, octyl sulfates, 2-ethylhexyl sulfates, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, ricinoleates, cetyl sulfates, and similar surfactants. Additional examples of surfactants can be found in CTFA Cosmetic Ingredient Handbook, J. M. Nikitakis, ed., The Cosmetic, Toiletry and Fragrance Association, Inc., Washington, D.C. (1988) (hereafter CTFA Handbook), pages 10-13, 42-46, and 87-94, incorporated herein by reference.

The compositions also can contain nonionic surfactants. Typically, a nonionic surfactant has a hydrophobic base, such as a long chain alkyl group or an alkylated aryl group, and a hydrophilic chain comprising a sufficient number (i.e., 1 to about 30) of ethoxy and/or propoxy moieties. Examples of classes of nonionic surfactants include ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, and mixtures thereof.

Exemplary nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof.

Numerous other nonionic surfactants are disclosed in McCutcheon's, at pages 1-246 and 266-272; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Handbook, at pages 86-94, each incorporated herein by reference.

In addition to anionic and nonionic surfactants, cationic, ampholytic, and amphoteric surfactants can be used in the compositions. Useful cationic surfactants include those having a structural formula

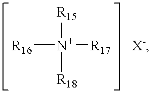

wherein R₁₅ is an alkyl group having about 12 to about 30 carbon atoms, or an aromatic, aryl, or alkaryl group having about 12 to about 30 carbon atoms; R₁₆, R₁₇, and R₁₈, independently, are selected from the group consisting of hydrogen, an alkyl group having 1 to about 22 carbon atoms, or aromatic, aryl, or alkaryl groups having from about 12 to about 22 carbon atoms; and X is a compatible anion, preferably selected from the group consisting of chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁₅, R₁₆, R₁₇, and R₁₈ also can contain ester and/or ether linkages, or hydroxy or amino group substituents (e.g., the alkyl groups can contain polyethylene glycol and polypropylene glycol moieties).

Preferably, R₁₅ is an alkyl group having about 12 to about 22 carbon atoms; R₁₆ is H or an alkyl group having 1 to about 22 carbon atoms; and R₁₇ and R₁₈, independently are H or an alkyl group having 1 to about 3 carbon atoms. More preferably, R₁₅ is an alkyl group having about 12 to about 22 carbon atoms, and R₁₆, R₁₇, and R₁₈ are H or an alkyl group having 1 to about 3 carbon atoms.

Other useful cationic surfactants include amino-amides, wherein in the above structure R₁₀ alternatively is R₁₉CONH—(CH₂)ₙ, wherein R₁₉ is an alkyl group having about 12 to about 22 carbon atoms, and n is an integer of 2 to 6, more preferably 2 to 4, and most preferably 2 to 3. Nonlimiting examples of these cationic surfactants include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereol

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof.

Additional quaternary ammonium salts include those wherein the C₁₂-C₃₀ alkyl carbon chain is derived from a tallow fatty acid or from a coconut fatty acid. The term “tallow” refers to an alkyl group derived from tallow fatty acids (usually hydrogenated tallow fatty acids), which generally has mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, and mixtures thereof. An example of a quaternary ammonium compound having an alkyl group with an ester linkage is ditallowyl oxyethyl dimethyl ammonium chloride.

Ampholytic surfactants, i.e., amphoteric and zwitterionic surfactants, can be broadly described as derivatives of secondary and tertiary amines having straight chain or branched aliphatic radicals, and wherein one of the aliphatic substituents contains from about 8 to about 18 carbon atoms and at least one of the aliphatic substituents contains an anionic water-solubilizing group, e.g., carboxy, sulfonate, or sulfate.

More particularly, one class of ampholytic surfactants include sarcosinates and taurates having the general structural formula 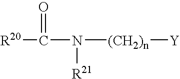
wherein R²⁰ is C₁₁-C₂₁, alkyl, R²¹ is hydrogen or C₁-C₂ alkyl, Y is CO₂M or SO₃M, M is an alkali metal, and n is a number 1 through 3.

Another class of ampholytic surfactants is the amide sulfosuccinates having the structural formula

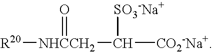

The following classes of ampholytic surfactants also can be used:

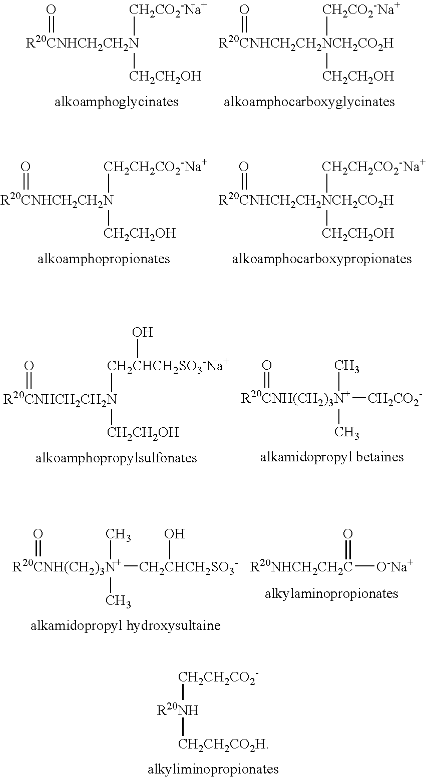

Additional classes of ampholytic surfactants include the phosphobetaines and the phosphitaines.

Specific, nonlimiting examples of ampholytic surfactants useful in the present invention are sodium coconut N-methyl taurate, sodium oleyl N-methyl taurate, sodium tall oil acid N-methyl taurate, sodium palmitoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamido-bis-(2-hydroxyethyl)propylsultaine, disodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, capryloamphocarboxypropionate, dihydroxyethyl tallow glycinate, cocamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, lauric myristic amido propyl monosodium phosphitaine, and mixtures thereof.

Useful amphoteric surfactants also include the amine oxides. Amine oxides have a general structural formula wherein the hydrophilic portion contains a nitrogen atom that is bound to an oxygen atom with a semipolar bond.

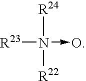

R²², R²³, and R²⁴ can be a saturated or unsaturated, branched, or unbranched alkyl or alkenyl group having 1 to about 24 carbon atoms. Preferred amine oxides contain at least one R group that is an alkyl chain of 8 to 22 carbon atoms. Nonlimiting examples of amine oxides include alkyl dimethyl amine oxides, such as decylamine oxide, cocamine oxide, myristamine oxide, and palmitamine oxide. Also useful are the alkylaminopropylamine oxides, for example, coamidopropylamine oxide and stearamidopropylamine oxide.

Nonlimiting examples of preferred surfactants utilized in a preconditioning composition include those selected from the group consisting of alkyl sulfates; alkyl ether sulfates; alkyl benzene sulfonates; alpha olefin sulfonates; primary or secondary alkyl sulfonates; alkyl phosphates; acyl taurates; alkyl sulfosuccinates; alkyl sulfoacetates; sulfonated fatty acids; alkyl trimethyl ammonium chlorides and bromides; dialkyl dimethyl ammonium chlorides and bromides; alkyl dimethyl amine oxides; alkylamidopropyl amine oxides; alkyl betaines; alkyl amidopropyl betaines; and mixtures thereof. More preferred surfactants include those selected from the group consisting of alkyl sulfates; alkyl ether sulfates; alkyl benzene sulfonates; alpha olefin sulfonates; primary or secondary alkyl sulfonates; alkyl dimethyl amine oxides; alkyl betaines; and mixtures thereof.

Additional cleansing compositions and surfactants are disclosed in U.S. Pat. Nos. 6,271,187 and 6,900,167, each incorporated herein by reference.

A preconditioning composition also can contain optional ingredients well known to persons skilled in the art. For example, the composition can contain a hydric solvent and/or a hydrotrope. The compositions also can contain other optional ingredients, such as dyes and fragrances, that are present in a sufficient amount to perform their intended function and not adversely affect the antibacterial efficacy of the preconditioning composition.

Classes of optional ingredients include, but are not limited to, dyes, fragrances, pH adjusters, thickeners, viscosity modifiers, buffering agents, foam stabilizers, antioxidants, foam enhancers, chelating agents, opacifiers, and similar classes of optional ingredients known to persons skilled in the art.

Specific classes of optional ingredients include alkanolamides as foam boosters and stabilizers; gums and polymers as thickening agents; inorganic phosphates, sulfates, and carbonates as buffering agents; EDTA and phosphates as chelating agents; and acids and bases as pH adjusters.

Preferred antibacterial compositions utilized in the preconditioning step provide a rapid and broad spectrum bacterial control and have an acidic to neutral pH. For example, an antibacterial composition that exhibits a log reduction against Gram positive bacteria (i.e., S. aureus) of at least 2 after 30 seconds of contact, a log reduction against Gram negative bacteria (i.e., E. coli) of at least 2.5 after 30 seconds of contact, and has pH of about 5 to about 8.

After application of the preconditioning composition to the skin, the preconditioning composition typically is rinsed from the skin, followed by application of the antiviral composition to the skin.

The following are general examples of preconditioning compositions that can be used in accordance with the present invention. Concentrated preconditioning compositions are disclosed in U.S. Pat. No. 6,271,187, incorporated herein by reference. Useful antibacterial compositions are disclosed in U.S. Pat. No. 6,107,261, incorporated herein by reference.

| | Weight % |
|---|---|
| Alpha-olefin sulfonate | 20% |
| Amphoteric surfactant | 5% |
| Alkanolamide | 2% |
| Perfume, Dye, Preservative | 0.5% |
| Citric acid | q.s. to pH 6.5 to 7 |
| Sodium chloride | q.s. to 3000 cps |
| Water | q.s. |
| Ethoxylated alcohol sulfate | 10% |
| Alcohol sulfate | 10% |
| Fatty acid monoethanolamide | 5% |
| Cocoamidopropylbetaine | 1.5% |
| Sodium chloride | 1.5% |
| EDTA salt | 0.2-0.5% |
| Glycerin | 1% |
| Water | to 100% |
| Dye, perfume, preservatives, and opacifier, as desired | |
| Adust pH to ≈7 with citric acid | |
| Viscosity, 73° F., cps | 7000 |
| Ethoxylated alcohol sulfate | 10% |
| Alcohol sulfate | 10% |
| Fatty acid monoethanolamide | 2% |
| Sodium chloride | 2% |
| EDTA salt | 0.2-0.5% |
| Glycerin | 1% |
| Water | to 100% |
| Dye, perfume, preservatives, and opacifier, as desired | |
| Adjust pH to ≈7 with citric acid | |
| Viscosity, 73° F., cps | 2200 |
| Ethoxylated alcohol sulfate | 15% |
| Cocoamidopropylbetaine | 5% |
| Fatty acid monoethanolamide | 5% |
| PEG (6000) distearate | 0.5% |
| Sodium chloride | 3% |
| EDTA salt | 0.2-0.5% |
| Glycerin | 1.0% |
| Water | to 100% |
| Dye, perfume, preservatives, and opacifier, as desired | |
| Adjust pH to ≈7 with citric acid | |
| Viscosity, 75° F., cps | 5400 |
| Ethoxylated alcohol sulfate | 15% |
| Fatty acid monoethanolamide | 5% |
| PEG (6000) distearate | 0.5% |
| Sodium chloride | 3% |
| EDTA salt | 0.2-0.5% |
| Glycerin | 1% |
| Water | to 100% |
| Dye, perfume, preservatives, and opacifier, as desired | |
| Adjust pH to ≈7 with citric acid | |
| Viscosity, 73° F., CPS | 4700 |
| Ethoxylated alcohol sulfate | 5% |
| Alpha olefin sulfonate | 12.5% |
| Fatty acid monoethanolamide | 2% |
| Cocoamidopropylbetaine | 5% |
| Sodium chloride | 5.5% |
| EDTA salt | 0.2-0.5 |
| Glycerin | 1 |
| Water | to 100% |
| Dye, perfume, preservatives, and opacifier, as desired | |
| Adjust pH to ≈7 with citric acid | |
| Viscosity, 73° F., cps | 4700 |

Virus Control Step

After the preconditioning step, an antiviral composition is applied to skin to impart a persistent virus control to the skin. The antiviral composition is allowed to contact the skin for a sufficient time to achieve a lowering of skin pH. The preconditioning step removes soil and sebum to enhance the skin pH-lowering effect, and antiviral efficacy, of the antiviral composition. The preconditioning step also standardizes skin pH to facilitate lowering of skin pH by the antiviral composition. If so designed, the antiviral composition can be rinsed from the skin after a contact time of about 30 seconds to 5 minutes. Preferably, the antiviral composition is not rinsed from, but is allowed to remain on, the skin. The antiviral composition is applied to the skin after the preconditioning step, and prior to an appreciable resoiling of the skin. Preferably, the antiviral composition is applied to the skin immediately after, or shortly after, the preconditioning step.

As illustrated in the following nonlimiting embodiments, a preferred antiviral composition used in the present method comprises: (a) about 25% to about 75%, by weight, of a disinfecting alcohol; (b) a virucidally effective amount of an organic acid; and (c) water. The antiviral compositions have a pH of less than about 5 to effectively reduce skin pH. In some embodiments, the antiviral composition contains an optional gelling agent and/or an optional active antibacterial agent.

The antiviral compositions typically are capable of forming an essentially continuous film or layer of nonvolatile composition ingredients on treated skin. The film or layer resists removal from the treated skin for several hours after application. In particular, an effective amount of the nonvolatile antiviral composition ingredients remain on treated skin after ten rinsings, and at least 50%, preferably at least 60%, and more preferably at least 70%, of the nonvolatile ingredients remains on treated skin after three rinsings. As used herein, “rinsing” means gently rubbing treated skin for 30 seconds under a moderate flow of tap water having a temperature of about 30° C. to about 40° C., then air drying the skin.

The antiviral composition provides an effective and persistent inactivation of nonenveloped viruses. Nonenveloped viruses include, but are not limited to, adenoviruses, papovaviruses, parvoviruses, bimaviruses, astroviruses, rotaviruses, caliciviruses (including Norwalk virus), and picornaviruses (including rhinovirus, polio virus, and hepatitis A virus). The compositions also effectively control and inactivate influenza viruses and noroviruses.

The antiviral compositions exhibit a log reduction against nonenveloped viruses, including acid-labile viruses, such as rhinovirus serotypes, of about 5 after 30 seconds contact, and a log reduction against these acid-labile viruses of at least 3 about five hours after contact, and at least about 2 about six to about eight hours after contact. The antiviral compositions exhibit a log reduction against noroviruses, and other caliciviruses, of about 3 after 30 seconds contact, and preferably a log reduction against these viruses of at least 2.5 about five hours, and at least 2 about six to about eight hours, after contact. The antiviral compositions also typically exhibit a log reduction against Gram positive bacteria of about 2 after 30 seconds contact. The antiviral composition further exhibits a log reduction against Gram negative bacteria of about 2.5 after 30 seconds contact. The antiviral compositions are mild, and it is not necessary to rinse or wipe the compositions from the skin.

The antiviral composition can further comprise additional optional ingredients disclosed hereafter, like hydrotropes, polyhydric solvents, gelling agents, pH adjusters, vitamins, dyes, skin conditioners, and perfumes. The antiviral compositions are free of intentionally-added cleansing surfactants, like anionic surfactants.

More particularly, antiviral compositions used in the present method contain about 25% to about 75%, by weight, of a disinfecting alcohol. Preferred embodiments of the present invention contain about 30% to about 75%, by weight, of a disinfecting alcohol. Most preferred embodiments contain about 30% to about 70%, by weight, of a disinfecting alcohol. As used herein, the term “disinfecting alcohol” is a water-soluble alcohol containing one to six carbon atoms, i.e., a C₁₋₆ alcohol. Disinfecting alcohols include, but are not limited to, methanol, ethanol, propanol, and isopropyl alcohol.

The antiviral composition also contains an organic acid in a sufficient amount to control and inactivate viruses and bacteria on a surface contacted by the antiviral composition. The organic acid acts synergistically with the disinfecting alcohol to provide a rapid control of nonenveloped viruses and/or bacteria, and provides a persistent viral control.

An organic acid is present in the antiviral composition in a sufficient amount such that the pH of the animate surface contacted by the composition is lowered to degree wherein a persistent viral control is achieved. This persistent viral control is achieved regardless of whether the composition is rinsed from, or allowed to remain on, the contacted surface. The organic acid remains at least partially undissociated in the composition, and remains so during application and optional rinsing.

Upon application to a surface, such as human skin, the pH of the surface is sufficiently lowered such that a persistent viral control is achieved. In preferred embodiments, a residual amount of the organic acid remains on the skin, even after an optional rinsing step, in order to impart a persistent viral control. In preferred embodiments, the organic acid remains on treated skin as an essentially continuous barrier film or layer. However, even if the organic acid is essentially completely rinsed from the surface, the surface pH has been sufficiently lowered to impart a viral control for at least 0.5 hour.

Typically, an organic acid is included in an antiviral composition in an amount of about 0.05% to about 15%, preferably about 0.1% to about 10%, and more preferably about 0.15% to about 6%, by weight of the composition. The total amount of organic acid is related to the class of organic acid used, and to the identity of the specific acid or acids used.

An organic acid included in the antiviral composition preferably does not penetrate the surface to which it is applied, e.g., remains on the skin surface as opposed to penetrating the skin and forms a layer or film on the skin, together with another nonvolatile composition ingredients, e.g., an optional gelling agent. The organic acid, therefore, preferably is a hydrophobic organic acid.

In one embodiment, the organic acid has a log P of less than one, and preferably less than 0.75, less than 0.5. In this embodiment, the disinfecting alcohol and organic acid act synergistically to provide an effective and persistent viral control.

In another embodiment, the organic acid has a log P of 1 or greater, for example, 1 to about 100. In this embodiment, the disinfecting alcohol and organic acid effectively control nonenveloped viruses and also act synergistically to control a broad spectrum of bacteria.

It is envisioned that, by incorporating a first organic acid having a log P of less than one and a second organic acid having a log P of 1 or greater into a present composition, the first and second organic acids act synergistically with the disinfecting alcohol to provide a persistent control of nonenveloped viruses and a broad spectrum bacteria control.

As used herein, the term “log P” is defined as the log of the water-octanol partition coefficient, i.e., the log of the ratio P_{w}/Pₒ, wherein P_{w} is the concentration of an organic acid in water and Pₒ is the concentration of the organic acid in octanol, at equilibrium and 25° C. The water-octanol coefficient can be determined by the U.S. Environmental Protection Agency Procedure, “OPPTS 830.7560 Partition Coefficient (n-Octanol/Water), Generator Column Method” (1996).

Organic acids having a log P less than one typically are water insoluble, e.g., have a water solubility of less than about 0.5 wt % at 25° C. Organic acids having a log P of one or greater typically are considered water soluble, e.g., have a water solubility of at least 0.5 wt %, at 25° C.

The organic acid can comprise a monocarboxylic acid, a polycarboxylic acid, a polymeric acid having a plurality of carboxylic, phosphate, sulfonate, and/or sulfate moieties, or mixtures thereof. In addition to acid moieties, the organic acid also can contain other moieties, for example, hydroxy groups and/or amino groups. In addition, an organic acid anhydride can be used in a composition of the present invention as the organic acid.

In one embodiment, the organic acid comprises a monocarboxylic acid having a structure RCO₂H, wherein R is C₁₋₆alkyl, hydroxyC₁₋₆alkyl, haloC₁₋₆alkyl, phenyl, or substituted phenyl. The alkyl groups can be substituted with phenyl groups and/or phenoxy groups, and these phenyl and phenoxy groups can be substituted or unsubstituted.

Nonlimiting examples of monocarboxylic acids useful in the present invention are acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof. Additional substituted benzoic acids are disclosed in U.S. Pat. No. 6,294,186, incorporated herein by reference. Examples of substituted benzoic acids include, but are not limited to, salicyclic acid, 2-nitrobenzoic acid, thiosalicylic acid, 2,6-dihydroxybenzoic acid, 5-nitrosalicyclic acid, 5-bromosalicyclic acid, 5-iodosalicyclic acid, 5-fluorosalicylic acid, 3-chlorosalicylic acid, 4-chlorosalicyclic acid, and 5-chlorosalicyclic acid.

In another embodiment, the organic acid comprises a polycarboxylic acid. The polycarboxylic acid contains at least two, and up to four, carboxylic acid groups. The polycarboxylic acid also can contain hydroxy or amino groups, in addition to substituted and unsubstituted phenyl groups.

Nonlimiting examples of polycarboxylic acids useful in the present invention include malonic acid, succinic acid, glutaric acid, adipic acid, terephthalic acid, phthalic acid, pimelic acid, suberic acid, azelaic acid, sebacic acid, fumaric acid, maleic acid, tartaric acid, malic acid, citric acid, maleic acid, aconitic acid, and mixtures thereof.

Anhydrides of polycarboxylic and monocarboxylic acids also are organic acids useful in the present compositions. Preferred anhydrides are anhydrides of polycarboxylic acids, e.g., phthalic anhydride. At least a portion of the anhydride is hydrolyzed to a carboxylic acid because of the pH of the composition. It is envisioned that an anhydride can be slowly hydrolyzed on a surface contacted by the composition, and thereby assist in providing a persistent antiviral activity.

In a third embodiment, the organic acid comprises a polymeric carboxylic acid, a polymeric sulfonic acid, a sulfated polymer, a polymeric phosphoric acid, or mixtures thereof. The polymeric acid has a molecular weight of about 500 g/mol to 10,000,000 g/mol, and includes homopolymers, copolymers, and mixtures thereof. The polymeric acid preferably is capable of forming a substantive film on a surface and has a glass transition temperature, T_{g}, of less than 25° C., preferably less than 20° C., and more preferably less than about 15° C. The glass transition temperature is the temperature at which an amorphous material, such as a polymer, changes from a brittle, vitreous state to a plastic state. The T_{g} of a polymer is readily determined by persons skilled in the art using standard techniques.

The polymeric acids are uncrosslinked or only very minimally crosslinked. The polymeric acids typically are prepared from ethylenically unsaturated monomers having at least one hydrophilic moiety, such as carboxyl, carboxylic acid anhydride, sulfonic acid, and sulfate. The polymeric acid can contain a comonomer, such as styrene or an alkene, to increase the hydrophobicity of the polymeric acid.

Examples of monomers used to prepare the polymeric organic acid include, but are not limited to:

(a) Carboxyl group-containing monomers, e.g., monoethylenically unsaturated mono- or polycarboxylic acids, such as acrylic acid, methacrylic acid, maleic acid, fumaric acid, crotonic acid, sorbic acid, itaconic acid, ethacrylic acid, α-chloroacrylic acid, α-cyanoacrylic acid, β-methlacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, citraconic acid, mesaconic acid, glutaconic acid, aconitic acid, tricarboxyethylene, and cinnamic acid;
(b) Carboxylic acid anhydride group-containing monomers, e.g., monoethylenically unsaturated polycarboxylic acid anhydrides, such as maleic anhydride; and

(c) Sulfonic acid group-containing monomers, e.g., aliphatic or aromatic vinyl sulfonic acids, such as vinylsulfonic acid, allylsulfonic acid, vinyltoluenesulfonic acid, styrenesulfonic acid, sulfoethyl (meth)acrylate, 2-acrylamido-2-methylpropane sulfonic acid, sulfopropyl (meth)acrylate, and 2-hydroxy-3-(meth)acryloxy propyl sulfonic acid.

The polymeric acid can contain other copolymerizable units, i.e., other monoethylenically unsaturated comonomers, well known in the art, as long as the polymer is substantially, i.e., at least 10%, and preferably at least 25%, acid group containing monomer units. To achieve the full advantage of the present invention, the polymeric acid contains at least 50%, and more preferably, at least 75%, and up to 100%, acid group containing monomer units. The other copolymerizable units, for example, can be styrene, an alkene, an alkyl acrylate, or an alkyl methacrylate. The polymeric acid also can be partially neutralized, which assists dispersion of the polymeric acid into a composition. However, a sufficient number of the acid groups remain unneutralized to reduce skin pH and impart a persistent antiviral activity.

A polymeric acid assists in forming a film or layer of residual organic acid on the skin, and assists further in forming a more continuous layer of residual organic acid on the skin. A polymeric acid typically is used in conjunction with a monocarboxylic acid and/or a polycarboxylic acid.

One preferred polymeric acid is a polyacrylic acid, either a homopolymer or a copolymer, for example, a copolymer of acrylic acid and an alkyl acrylate and/or alkyl methacrylate. Another preferred polymeric acid is a homopolymer or a copolymer of methacrylic acid.

Exemplary polymeric acids useful in the present invention include, but are not limited to:

| | (CARBOPOL 910, |
|---|---|
| | 934, 934P, 940, 941, |
| | ETD 2050; ULTREZ |
| | 10, 21) |
| | (CARBOPOL ETD |
| Carbomers | 2050) |
| Acrylates/C20-30 Alkyl Acrylate Crosspolymer | (ULTREZ 20) |
| Acrylates/Beheneth 25 Methacrylate Copolymer | (ACULYN 28) |
| Acrylates/Steareth 20 Methacrylate Copolymer | (ACULYN 22) |
| Acrylates/Steareth 20 Methacrylate Crosspolymer | (ACULYN 88) |
| Acrylates Copolymer | (CAPIGEL 98) |
| Acrylates Copolymer | (AVALURE AC) |
| Acrylates/Palmeth 25 Acrylate Copolymer | (SYNTHALEN 2000) |
| Ammonium Acrylate Copolymers | |
| Sodium Acrylate/Vinyl Alcohol Copolymer | |
| Sodium Polymethacrylate | |
| Acrylamidopropyltrimonium Chloride/ | |
| Acrylates Copolymer | |
| Acrylates/Acrylamide Copolymer | |
| Acrylates/Ammonium Methacrylate Copolymer | |
| Acrylates/C10-30 Alkyl Acrylate Crosspolymer | |
| Acrylates/Diacetoneacrylamide Copolymer | |
| Acrylates/Octylacrylamide Copolymer | |
| Acrylates/VA Copolymer | |
| Acrylic Acid/Acrylonitrogens Copolymer | |

In a preferred embodiment of the antiviral composition, the organic acid comprises one or more polycarboxylic acid, e.g., citric acid, malic acid, tartaric acid, or a mixture of any two or all three of these acids, and a polymeric acid containing a plurality of carboxyl groups, for example, homopolymers and copolymers of acrylic acid or methacrylic acid.

The antiviral composition also contains water as a carrier and optional ingredients well known to persons skilled in the art. The optional ingredients are present in a sufficient amount to perform their intended function and not adversely affect the antibacterial efficacy of the composition, and in particular not adversely affect the synergistic effect provided by the disinfecting alcohol and organic acid. Optional ingredients typically are present, individually or collectively, from 0% to about 50%, by weight of the composition.

Classes of optional ingredients include, but are not limited to, hydrotropes, polyhydric solvents, antibacterial agents, gelling agents, dyes, fragrances, pH adjusters, thickeners, viscosity modifiers, chelating agents, skin conditioners, emollients, preservatives, buffering agents, antioxidants, chelating agents, opacifiers, and similar classes of optional ingredients known to persons skilled in the art.

A hydrotrope, if present at all, is present in an amount of about 0.1% to about 30%, and preferably about 1% to about 20%, by weight of the composition. A hydrotrope is a compound that has an ability to enhance the water solubility of other compounds. A hydrotrope utilized in the present invention lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A polyhydric solvent, if present at all, is present in an amount of about 0.1% to about 30%, and preferably about 5% to about 30%, by weight of the composition. In contrast to a disinfecting alcohol, a polyhydric solvent contributes minimally, if at all, to the antibacterial efficacy of the present composition. The term “polyhydric solvent” as used herein is a water-soluble organic compound containing two to six, and typically two or three, hydroxyl groups. The term “water-soluble” means that the polyhydric solvent has a water solubility of at least 0.1 g of polyhydric solvent per 100 g of water at 25° C. There is no upper limit to the water solubility of the polyhydric solvent, e.g., the polyhydric solvent and water can be soluble in all proportions.

The term polyhydric solvent, therefore, encompasses water-soluble diols, triols, and polyols. Specific examples of hydric solvents include, but are not limited to, ethylene glycol, propylene glycol, glycerol, diethylene glycol, dipropylene glycol, tripropylene glycol, hexylene glycol, butylene glycol, 1,2,6-hexanetriol, sorbitol, PEG-4, and similar polyhydroxy compounds.

The antiviral composition can contain an active antibacterial agent. The antibacterial agent is present in an antiviral composition is the same as disclosed above for a preconditioning composition. Useful antibacterial agents are discussed above in connection with the preconditioning composition. Antiviral compositions containing a phenolic antibacterial agent also are effective in controlling and inactivating noroviruses.

The antiviral compositions also can contain, if at all, about 0.01% to about 5%, by weight, and preferably 0.10% to about 3%, and more preferably about 0.25% to about 2.5%, by weight, of an optional gelling agent. The term “gelling agent” as used here and hereafter refers to a compound capable of increasing the viscosity of a water-based composition, or capable of converting a water-based composition to a gel or semisolid. The gelling agent can be organic in nature, for example, a natural gum or a synthetic polymer, or can be inorganic in nature. The antiviral compositions typically contain a sufficient amount of gelling agent such that the composition is a viscous liquid, gel, or semisolid that can be easily applied to, and rubbed on, the skin or other surface. Persons skilled in the art are aware of the type and amount of gelling agent to include in the composition to provide the desired composition viscosity or consistency.

As previously stated, the present compositions preferably are free of a surfactant. A surfactant typically is not intentionally added to a present antibacterial composition, but may be present in an amount of 0% to about 0.5%, by weight, because a surfactant may be present in a commercial form of a gelling agent to help disperse the gelling agent in water. A surfactant also may be present as an additive or by-product in other composition ingredients.

The following are nonlimiting examples of gelling agents that can be used in the present invention. In particular, the following compounds, both organic and inorganic, act primarily by thickening or gelling the aqueous portion of the composition: acacia, agar, algin, alginic acid, ammonium alginate, ammonium chloride, ammonium sulfate, amylopectin, attapulgite, bentonite, C₉₋₁₅ alcohols, calcium acetate, calcium alginate, calcium carrageenan, calcium chloride, caprylic alcohol, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, fruit pectin, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-2M, PEG-5M, polyvinyl alcohol, potassium alginate, potassium carrageenan, potassium chloride, potassium sulfate, potato starch, propylene glycol alginate, sodium carboxymethyl dextran, sodium carrageenan, sodium cellulose sulfate, sodium chloride, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, polyvinylpyrrolidone and derivatives thereof, vinyl ether derivatives (methyl vinyl ether, ethyl vinyl ether, butyl vinyl ether, isobutyl vinyl ether, polymethyl vinyl ether/maleic acid), quaternized vinylpyrrolidone/quaternized dimethylamino ethyl pyrrolidone-based polymers and methacrylate copolymers, vinylcaprolactam/vinylpyrrolidone dimethylamino ethylmethacrylate polymers, vinylpyrrolidone/dimethyl amino ethylmethacrylate copolymers, acid stable and naturally occurring derivatives of guar and modified guar, modified or substituted xanthan, carboxypropyl cellulose, and mixtures thereof.

The following additional nonlimiting examples of gelling agents act primarily by thickening the nonaqueous portion of the composition:

abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, triisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

Exemplary gelling agents present in an antiviral composition include, but are not limited to,

| | |
|---|---|
| Polyethylene Glycol & Propylene Glycol & Water | (ACULYN 44) |
| Ammonium Acrylatedimethyltaurate/VP Copolymer | (ARISTOFLEX AVC) |
| Glyceryl Stearate & PEG 100 Stearate | (ARLACEL 165) |
| Polyethylene(2)Stearyl Ether | (BRIJ 72) |
| Polyoxyethylene(21)Stearyl Ether | (BRIJ 721) |
| Silica | (CAB-O-SIL) |
| Polyquaternium 10 | (CELQUAT CS230M) |
| Cetyl Alcohol | |
| Cetearyl Alcohol & Cetereth 20 | (COSMOWAX P) |
| Cetearyl Alcohol & Dicetyl Phosphate & Ceteth-10 Phosphate | (CRODAFOS CES) |
| Ceteth-20 Phosphate & Cetearyl Alcohol & Dicetyl Phosphate | (CRODAFOS CS-20 Acid) |
| Cetearyl Alcohol & Cetereth 20 | (EMULGADE NI 1000) |
| Sodium Magnesium Silicate | (LAPONITE XLG) |
| Cetyl Alcohol & Stearyl Alcohol & Stearalkonium Chloride & Dimethyl | (MACKADET CBC) |
| Stearamine & Lactic Acid | |
| Cetearyl Alcohol & Stearamidopropyldimethylamine & | (MACKERNIUM |
| Stearamidopropylalkonium Chloride | Essential) |
| Stearalkonium Chloride | (MACKERNIUM SDC-85) |
| Cetearyl Alcohol & Stearamidopropyldimethylamine & | (MACKERNIUM Ultra) |
| Stearamidopropylalkonium Chloride & Silicone Quaternium 16 | |
| Cetearyl Alcohol & Cetearyl Glucoside | (MONTANOV 68EC) |
| Hydroxyethylcellulose | (NATROSOL 250 HHR |
| | CS) |
| Polyquaternium-37 & Mineral Oil & Trideceth-6 | (SALCARE SC 95) |
| Polyquaternium-32 & Mineral Oil & Trideceth-6 | (SALCARE SC 96) |
| Stearic Acid | |
| Cetyl Hydroxyethylcellulose | (NATROSOL Plus 330 CS) |
| Polyvinyl Alcohol, PVP-K30, Propylene Glycol | |
| Stearic Acid, Behenyl Alcohol, Glyceryl Stearate, Lecithin, C12-16 | (PROLIPID 141) |
| Alcohols, Palmic Acid | |
| Beeswax | (saponified beeswax) |
| Beeswax | (synthetic beeswax) |
| Water, Beeswax, Sesame Oil, Lecithin, Methyl paraben | (beesmilk) |
| Polyquaternium 10 | (CELQUAT SC240C) |
| Sodium Acrylate/Sodium Acrylodimethyl Taurate Copolymer & | (SIMULGEL EG) |
| Isohexadecane & Polysorbate 80 | |
| Polyquaternium 44 | (LUVIQUAT Care) |

Other specific classes of optional ingredients include inorganic phosphates, sulfates, and carbonates as buffering agents; EDTA and phosphates as chelating agents; and acids and bases as pH adjusters.

Examples of preferred classes of optional basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of preferred classes of optional acidic pH adjusters are the mineral acids. Nonlimiting examples of mineral acids are hydrochloric acid, nitric acid, phosphoric acid, and sulfuric acid.

The composition also can contain a cosolvent or a clarifying agent, such as a polyethylene glycol having a molecular weight of up to about 4000, methylpropylene glycol, an oxygenated solvent of ethylene, propylene, or butylene, or mixtures thereof. The cosolvent or clarifying agent can be included as needed to impart stability and/or clarity to the composition and may be present in the residual film or layer of the composition on a treated surface.

The pH of the antiviral composition is about 5 or less, and preferably less than about 4.5 at 25° C. To achieve a full advantage, the pH is less than about 4. Typically, the pH of an antiviral composition is about 2 to less than about 5, and preferably about 2.5 to about 4.5.

The pH of the antiviral composition is sufficiently low such that at least a portion of the organic acid is in the protonated form. The organic acid then has the capability of lowering skin pH to provide an effective viral control, without irritating the skin. The organic acid also deposits on the skin, can form an essentially continous barrier layer on the skin, and resists removal by rinsing, to provide a persistent antiviral effect.

The following is an example of an antiviral composition, which is capable of reducing skin pH, and providing a persistent antiviral activity to treated skin.

Sample 2

| Material | Percent (by weight) |
|---|---|
| Ethanol | 70.0 |
| Deionized water | 19.8 |
| ULTREZ ® 20¹⁾ | 1.0 |
| Isopropyl palmitate | 1.0 |
| Mineral oil | 1.0 |
| DC 200 silicone fluid | 1.0 |
| Cetyl alcohol | 1.0 |
| Citric acid | 2.0 |
| Malic acid | 2.0 |
| GERMABEN II²⁾ | 1.0 |
| Triethanolamine | 0.005 |
| | 100.0 |

¹⁾Acrylate/C10-30 Alkyl Acrylate Crosspolymer;
²⁾Preservative containing propylene glycol, diazolidinyl urea, methylparaben, and propylparaben.

The pH of the composition was 3.1.

To demonstrate the new and unexpected results provided by the method of the present invention, the following tests were conducted, and the ability of the method to control rhinovirus, was determined. The weight percentage listed in each of the following examples represents the actual, or active, weight amount of each ingredient present in the composition. The compositions are prepared by blending the ingredients, as understood by those skilled in the art and as described below.

The following methods are used in the preparation and testing of the examples:

Antiviral Residual Efficacy Test

References: S. A. Sattar, Standard Test Method for Determining the Virus-Eliminating Effectiveness of Liquid Hygienic Handwash Agents Using the Fingerpads of Adult Volunteers, Annual Book of ASTM Standards. Designation E1838-96, incorporated herein by reference in its entirety, and referred to as “Sattar I”; and S. A. Sattar et al., Chemical Disinfection to Interrupt Transfer of Rhinovirus Type 14 from Environmental Surfaces to Hands, Applied and Environmental Microbiology, Vol. 59, No. 5, May, 1993, pp. 1579-1585, incorporated herein by reference in its entirety, and referred to as “Sattar II.”

The method used to determine the Antiviral Index of the present invention is a modification of that described in Sattar I, a test for the virucidal activity of liquid hand washes (rinse-off products). The method is modified in this case to provide reliable data for leave-on products.

Modifications of Sattar I include the product being delivered directly to the skin as described below, virus inoculation of the fingerpads as described below, and viral recovery using ten-cycle washing. The inoculated skin site then is completely decontaminated by treating the area with 70% dilution of ethanol in water.

Procedure:

Ten-minute Test:

Subjects (5 per test product) initially wash their hands with a nomedicated soap, rinse the hands, and allow the hands to dry.

The hands then are treated with 70% ethanol and air dried.

Test product (1.0 ml) is applied to the hands, except for the thumbs, and allowed to dry.

About 10 minutes (±30 seconds) after product application, 10 μl of a Rhinovirus 14 suspension (ATCC VR-284, approximately 1×10⁶ PFU (plaque-forming units)/ml) is topically applied using a micropipette to various sites on the hand within a designated skin surface area known as fingerpads. At this time, a solution of rhinovirus also is applied to the untreated thumb in a similar manner.

After a dry-down period of 7-10 minutes, the virus then is eluted from each of the various skin sites with 1 ml of eluent (Earle's Balanced Salt Solution (EBSS) with 25% Fetal Bovine Serum (FBS)+1% pen-strep-glutamate), washing 10 times per site.

The inoculated skin site then is completely decontaminated by rinsing the area with 70% ethanol. Viral titers are determined using standard techniques, i.e., plaque assays or TCID₅₀ (Tissue Culture Infectious Dose).

One-hour test:

Subjects are allowed to resume normal activities (with the exception of washing their hands) between the 1-hour and 3-hour timepoints. After one hour, a rhinovirus suspension is applied to and eluted from designated sites on the fingerpads exactly as described in above for the 10-minute test.

### EXAMPLE 1

The standardization of skin pH attributed to a preconditioning step was demonstrated by applying Dial Complete, an antibacterial cleanser, to the skin. In one experiment, commercial Dial Complete (pH 5.8) was applied to the fingertips of a test subject (Test Subject 1). In a second experiment, Dial Complete adjusted to pH 3.8 was applied to the fingertips of a second test subject (Test Subject 2). In each test, a baseline skin pH was measured. Skin pH was measured again after the applied antibacterial cleanser dried on the skin. The results are summarized below.

| | | Skin pH after |
|---|---|---|
| | Baseline Skin pH | Preconditioning Step |
| Test subject 1 | 4.82 | 5.60 |
| Test subject 2 | 5.08 | 5.32 |

The skin pH data shows that for both preconditioning compositions, i.e., pH 3.8 and pH 5.8, the skin pH was standardized to 5.3 to 5.6. In addition, the skin was cleansed of soil and sebum. Although skin pH was raised after this preconditioning step, the skin pH remained a pH unit less than 6.5, and, therefore, conditioned the skin for an effective application of an antiviral composition.

### EXAMPLE 2

The following antiviral compositions first were prepared.

| Ingredient | Composition A¹⁾ | Composition B |
|---|---|---|
| Ethyl alcohol | 62% | 62% |
| L-Malic acid | 2% | 2% |
| Citric acid | 2% | 2% |
| ULTREZ 20²⁾ | 1% | — |
| NATROSOL 250 HHX³⁾ | — | 1% |
| Water | q.s. | q.s. |
| pH | 3.5 | 3.5 |

¹⁾Percent by weight, as active ingredient;
²⁾Acrylates C20-30 Alkyl Acrylate Crosspolymer; and
³⁾hydroxyethylcellulose.

In this example, three milliliters of antiviral Composition A or B was applied to cover the hands of test subjects. In one group, the hands were preconditioned using a preconditioning composition. In a second group, the antiviral composition was applied to “unconditioned” hands. The composition used in the preconditioning step was a commercially available, mild baby soap containing cocamidopropyl betaine, PEG-80 sorbitan laurate, sodium lauryl sulfate, PEG-150 distearate, tetrasodium EDTA, sodium chloride, polyquatemium-10, quatemium-15, citric acid, fragrance, and water, had a pH of between 6 and 7. The test subjects were allowed to use their hands in a normal fashion following application of the antiviral composition, then the fingers were challenged with Rhinovirus Type 39 after three hours. The tables below show that the efficacy of both Compositions A and B were improved when a preconditioning step was performed prior to application of the antiviral composition. In particular, the percent of hands found positive with rhinovirus decreased from 100% to 75% for Composition B after a preconditioning step, and from 33% to 12% for Composition A. The logio recovery of virus also was reduced for both Compositions A and B indicating that components of the antiviral composition were better retained and available to act against viruses on preconditioned hands.

| | | Percent of Hands Positive | | | | Avg. log₁₀ Virus | |
|---|---|---|---|---|---|---|---|
| | | Rhinovirus 39 | | | | Recovery | |
| | Time | Composition B | | Composition A | | Composition B | Composition A |
| Normal Use | | | | | | | |
| No preconditioning | | | | | | | |
| n = 3 | 3 hrs. | 100% | 6/6 | 33% | 2/6 | 1.47 | 0.86 |
| Normal Use | | | | | | | |
| With | | | | | | | |
| preconditioning | | | | | | | |
| n = 4 | 3 hrs. | 75% | 6/8 | 12% | 1/8 | 0.89 | 0.39 |

### EXAMPLE 3

The following table shows that the initial skin pH of the hands was reduced using Compositions A and B following a preconditioning step with the neutral preconditioning composition of Example 2. Reducing skin pH has been shown to increase antiviral efficacy against rhinovirus. Two pumps, or about one to about two liquid ounces, of the preconditioning composition from a container was dispensed into the wet palms of a test subject. The hands were rubbed together in a normal hand-washing motion for 15 seconds, then the hands were rinsed for 30 seconds under warm water. An antiviral Composition A or B then was dispensed onto the hands in metered doses (about one ounce) and rubbed onto the hands.

Normal Use (no conditioning wash)

| | Composition B | | Composition A | |
|---|---|---|---|---|
| | Starting pH | Starting pH | Starting pH | Starring pH |
| Time | No Conditioning | Conditioned | No Conditioning | Conditioned |
| 3 hrs. | 3.38 | 3.28 | 3.27 | 3.04 |

### EXAMPLE 4

The following antiviral composition, which is capable of reducing skin pH, was prepared and applied to the fingerpads of human volunteers:

Sample 4

| Material | Percent (by weight) |
|---|---|
| Ethanol | 70.0 |
| Deionized water | 19.8 |
| ULTREZ ® 20¹⁾ | 1.0 |
| Isopropyl Palmitate | 1.0 |
| Mineral oil | 1.0 |
| DC 200 silicone fluid | 1.0 |
| Cetyl alcohol | 1.0 |
| Citric acid | 2.0 |
| Malic acid | 2.0 |
| GERMABEN II²⁾ | 1.0 |
| Triethanolamine | 0.05 |
| | 100.0 |

¹⁾Acrylate/C10-30 Alkyl Acrylate Crosspolymer;
²⁾Preservative containing propylene glycol, diazolidinyl urea, methylparaben, and propylparaben.

The pH of Sample 4 was 3.1.

In the test, Sample 4 was applied to the fingerpads of all fingers, except the thumbs, of eight volunteers. The thumbs were control sites. The volunteers were divided into fours groups of two each. Each group I-IV then was challenged at a predetermined time with rhinovirus titer on all the fingerpads of each hand to determine the time-dependent efficacy of the test composition. At the time appropriate for each group, the skin pH of the fingerpads also was measured to determine the time course of skin pH in response to the test composition. The predetermined test time for rhinoviral challenge and skin pH measurement for each group I-IV were 5 minutes, 1 hour, 2 hours, and 4 hours, respectively. The following table summarizes the average log (rhinoviral titer inoculum), average skin pH, and average log (rhinoviral titer recovered) from the test fingerpads of the volunteers in the study, organized by group.

| | Initial skin pH | | Log | |
|---|---|---|---|---|
| | after | | [Inoculum | |
| | application | Skin pH at test | Titer] | Log [Recovered |
| Group | (average) | time (average) | (average) | Titer] (average) |
| I | 3.0 | 3.0 | 3.9 | 0.23 |
| II | 2.8 | 3.4 | 4.0 | 0.23 |
| III | 3.0 | 3.8 | 3.8 | 0.23 |
| IV | 3.0 | 3.8 | 4.3 | 0.23 |

The data for each group (i.e., different time points) shows that the average recovered rhinoviral titer is less than 1 virus particle, or below the detection limit of the test. This data illustrates the efficacy of the present method after 4 hours and further demonstrates that a skin pH of less than about 4 is effective at completely eliminating a virus challenge. The combination of citric acid, malic acid, and polymeric acid (i.e., ULTREZ® 20) provided a residual barrier layer of organic acids on the fingerpads, which enhanced the persistent antiviral activity of the composition.

### EXAMPLE 5

The clean fingerpads of test subjects were treated with the following compositions. Baseline skin pH readings were measured from the fingerpads prior to treatment with the compositions. Skin pH measurements also were taken immediately after the composition dried on the fingerpads, then again after four hours.

| | | | | | % |
|---|---|---|---|---|---|
| | | Average | Average | Viral | Hands |
| | | Skin pH | Skin pH | Log 10 | with |
| Sample | Composition (by wt %) | (T = 0) | (T = 4 hr) | Reduction | Virus |
| A | 2% citric acid, 2% malic | 2.81 | 3.23 | >3 log₁₀ | 0 |
| | acid, 62% ETOH, 1.25% | | | | |
| | hydroxyethylcellulose | | | | |
| B | 2% citric acid, | 2.64 | 3.03 | >3 log₁₀ | 0 |
| | 2% tartaric acid, | | | | |
| | 62% ETOH, 1.25% | | | | |
| | hydroxyethylcellulose | | | | |
| C | 2% malic acid, 2% tartaric | 2.66 | 2.94 | >3 log₁₀ | 0 |
| | acid, 62% ETOH, 1.25% | | | | |
| | hydroxyethylcellulose | | | | |
| D | 62% ETOH, 1.25% | 5.53 | 5.13 | <0.5 log₁₋ | 100 |
| | hydroxyethylcellulose | | | | |
| E | 2% citric acid, 2% malic | 2.90 | 3.72 | >3 log₁₀ | 0 |
| | acid, 70% ETOH, 1% | | | | |
| | polyacrylic acid | | | | |
| F | 70% ETOH, 1% polyacrylic | 4.80 | 5.16 | 2.0 log₁₀ | 100 |
| | acid | | | | |
| G | 70% ETOH, 1.25% | 5.3 | 5.25 | <0.5 log₁₀ | 100 |
| | hydroxyethylcellulose | | | | |

¹⁾ETOH is ethanol

Four hours after treatment of the fingerpads with Samples A-G, Rhinovirus 39 at a titer of 1.3×10³ pfu (plaque forming units) was applied to fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 39, i.e., a greater than 3 log reduction, was achieved using the acid-containing compositions containing a mixture of two of citric acid, malic acid, and tartaric acid. The presence of hydroxyethylcellulose or polyacrylic acid assisted in forming a more continuous film or layer of organic acids on the treated fingerpads, which in turn enhanced the persistent antiviral activity of the compositions.

### EXAMPLE 6

The clean fingerpads of test subjects were treated with the following composition. Baseline skin pH readings were measured from the fingerpads prior to treatment with the compositions. Skin pH measurements also were taken immediately after the composition dried on the fingerpads.

Immediately after treatment of the fingerpads with the composition, Rhinovirus 14 at a titer of 1.4×10⁴ pfu (plaque forming units) was applied to the fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 14 was achieved with the acid-containing composition resulting in a 4 log reduction.

| | | | Viral Log 10 | |
|---|---|---|---|---|
| | Composition | Solution | Reduction | % Hands |
| Sample | (by wt %) | pH | 30 seconds | with Virus |
| A | 2% citric acid, 2% | 3.10 | 4 log | 0 |
| | malic acid, 70% | | | |
| | ETOH, 1% | | | |
| | polyacrylic acid | | | |

### EXAMPLE 7

The following compositions were prepared to test the effect of organic acids and organic acid blends on skin pH and antiviral efficacy.

| | | Average | Average | Viral |
|---|---|---|---|---|
| | | Skin pH | Skin pH | Log10 |
| Sample | Composition (by wt %) | (T = 0) | (T = 2 hr) | Reduction |
| A | 4% citric acid in 70% | 2.97 | 3.64 | >3 log₁₀ |
| | ethanol/water | | | |
| B | 4% malic acid in 70% | 2.91 | 3.94 | >3 log₁₀ |
| | ethanol/water | | | |
| C | 2% citric acid and 2% malic | 2.99 | 3.38 | >3.log₁₀ |
| | acid in 70% ethanol/water | | | |
| D | 4% tartaric acid in 70% | 2.56 | 3.0 | >3 log₁₀ |
| | ethanol/water | | | |

The clean fingerpads of the test subjects were treated with Samples A-D. Baseline skin pH readings were measured from the fingerpads prior to treatment with a composition. Skin pH measurements also were taken immediately after the composition dried on the fingerpads, and again after two hours.

All Samples A-D suppressed skin pH to below 4 for two hours. The combination of citric acid and malic acid (Sample C) maintained a lower pH at two hours than the same acids used singly (Samples A and B). The 4% tartaric acid composition (Sample D) showed the greatest suppression of skin pH.

Two hours after treatment of the fingerpads with the solutions,
Rhinovirus 39 at a titer of 4×10⁴ pfu was applied to fingerpads. The virus was dried on the fingerpads for 10 minutes, then the fingerpads were rinsed with a viral recovery broth containing 75% EBSS and 25% FBS with 1× antibiotics. The sample was serially diluted in viral recovery broth and plated onto H1-HeLa cells. Titers were assayed as per the plaque assay. Complete inactivation of Rhinovirus 39 was achieved resulting in a greater than 3 log reduction.

The following examples illustrate that polymeric acids, and especially an acrylic acid homopolymer or copolymer, in the presence of alcohol impart antiviral efficacy. The polymeric acids have a low pH and good substantivity to skin, which effectively maintains a low skin pH over time, and helps provide a persistent antiviral efficacy. The polymeric acids also help provide an essentially continuous layer or film of an organic acid on treated surfaces, which in turn enhances the persistent antiviral activity of the composition.

A synergistic effect on the lowering of skin pH was demonstrated with using acrylic acid-based polymer in the presence of alcohol. However, an acrylic acid-based polymer in the absence of an alcohol did not maintain a reduced skin pH to the same degree over time. Importantly, skin pH reduction is less dependent on composition pH when a polymeric acid is used in conjunction with an alcohol.

A synergistic effect on a rapid and persistent antiviral activity also is demonstrated when an acrylic acid-based polymer is used in conjunction with polycarboxylic acids. It has been found that utilizing a low amount of a polymeric acid (e.g., about 0.1% to about 2%, by weight) together with a polycarboxylic acid, like citric acid, malic acid, tartaric acid, and mixtures thereof, enhances the antiviral activities of the polycarboxylic acids. This synergistic effect allows a reduction in the polycarboxylic acid concentration in an antiviral composition, without a concomitant decrease in antiviral efficacy. This reduction in polycarboxylic acid concentration improves composition mildness by reducing the irritation potential of the composition. It is theorized, but not relied upon herein, that the polymeric acid assists in forming a residual barrier film or layer of organic acids on a treated surface, which enhance the persistent antiviral activity of the composition.

### EXAMPLE 8

A composition containing a polyacrylic acid (1% by wt), i.e., ULTREZ 20, available from Noveon Europe, was prepared in 70% aqueous ethanol and in water. Each composition (1.8 ml) was applied to the thumb, index, and middle fingers of a test subject. Skin pH readings were measured prior to treatment (baseline), immediately after the fingers were dry, and again after two hours. The average skin pH readings are summarized below.

| | | | | Viral |
|---|---|---|---|---|
| | Average skin pH | | | log 10 |
| | Baseline | T = 0 | T = 2 hrs. | reduction |
| 70% ethanol | 5.65 | 5.3 | 5.2 | <0.2 |
| Polyacrylic acid (1%) (70% | 5.63 | 4.4 | 4.5 | 1.8 |
| aqueous ethanol) | | | | |
| Polyacrylic acid (1%) (water) | 5.64 | 4.5 | 4.7 | 1.5 |

The polyacrylic acid suppressed skin pH to about 4.5 initially, and skin pH remains under 5 after two hours. The composition with ethanol suppressed skin pH slightly lower (4.4) than the composition free of ethanol (4.5). This result suggests a synergistic effect on lowering skin pH when a polyacrylic acid is applied with ethanol.

Two hours after treatment of the fingerpads with the above compositions, Rhinovirus 39 was applied to the fingerpads that had been treated at a titer of 9.8×10² pfu. The virus was dried on the fingerpads for 10 minutes, then the fingerpads were rinsed with viral recovery broth. The broth was serially diluted in viral recovery broth and plated onto H1-HeLa cells. Titers were assayed as per the plaque assay. Both compositions reduced the viral titer. However, the composition containing ethanol exhibited slightly greater efficacy against Rhinovirus by reducing the titer by 1.8 log versus 1.5 log for the composition without ethanol.

This data illustrates that polyacrylic acid suppresses skin pH resulting in antiviral efficacy. The data also illustrates that polyacrylic acid and ethanol act synergistically to lower skin pH, thus resulting in a greater efficacy against rhinovirus.

To demonstrate this efficacy, the following eight compositions were prepared, wherein solutions containing a polyacrylic acid (with and without ethanol) were buffered to a pH of about 4.5, 5.0, 5.5, or 6.0.

| | | | Avg. | |
|---|---|---|---|---|
| | | Solution | Skin Ph | Viral log₁₀ |
| Sample | Composition (by wt %) | pH | 2 hrs. | Reduction |
| A | 1% ULTREZ 20/70% ethanol | 4.54 | 4.52 | >2 log₁₀ |
| B | 1% ULTREZ 20/70% ethanol | 5.10 | 4.87 | >2 log₁₀ |
| C | 1% ULTREZ 20/70% ethanol | 5.54 | 4.41 | >2 log₁₀ |
| D | 1% ULTREZ 20/70% ethanol | 6.17 | 4.32 | >2 log₁₀ |
| E | 1% ULTREZ 20 | 4.57 | 4.93 | <1 log₁₀ |
| F | 1% ULTREZ 20 | 5.12 | 5.46 | <1 log₁₀ |
| G | 1% ULTREZ 20 | 5.55 | 5.33 | <1 log₁₀ |
| H | 1% ULTREZ 20 | 6.32 | 5.70 | <1 log₁₀ |

The effect of the eight compositions on both skin pH and viral efficacy was tested. Each composition (1.8 ml) was applied to the thumb, index, and middle fingers of a test subject. Skin pH readings were measured prior to treatment (baseline), immediately after the product had dried, and again after two hours.

The skin pH data indicated that a polyacrylic acid and ethanol function synergistically to suppress skin pH because each composition containing ethanol in combination with the polyacrylic acid suppressed skin pH to a lower value than compositions free of ethanol. Compositions containing ethanol and polyacrylic acid lowered skin pH to between pH 4 and 5 independent of the solution pH. In contrast, compositions free of ethanol suppress the skin pH only to between pH 5-6 and the final skin pH is similar to the solution pH.

To test the viral efficacy of the above compositions, Rhinovirus 39 at a titer of 1.7×10³ pfu was applied to the fingerpads after two hours. The virus dried for 10 minutes, eluted and diluted serially in viral recovery broth. Samples were plated on H1-HeLa cells, and virus titer was assayed as per the plaque assay method. The compositions containing ethanol in combination with polyacrylic acid had a greater than 2 log reduction in viral titers, whereas compositions free of ethanol exhibited a less than 1 log reduction in viral titers. Therefore, a synergism exists between polyacrylic acid and ethanol in reducing skin pH, which provides greater antiviral efficacy against rhinovirus. It is theorized, but not relied upon herein, that the ethanol helps provide a more continuous film or layer of the organic acid on the skin, for example, by reducing the surface tension of the composition for a more even and uniform application of the composition to a surface, and particularly skin.

### EXAMPLE 9

The following compositions were prepared to further illustrate the antiviral efficacy provided by a polyacrylic acid.

| | | | | % |
|---|---|---|---|---|
| | | | Avg. | Hands |
| | Composition (by wt %) | Solution | Skin pH | with |
| Sample | Thickeners | pH | 2 hrs. | Virus |
| A | 1% polyacrylic acid | 4.21 | 4.7 | 63% |
| B | 5.5% CRODAFOS Acid¹⁾ | 5.41 | 5.0 | 100% |
| C | 1.25% NATROSOL 250 HHR | 6.32 | 5.3 | 100% |
| | CS²⁾ | | | |

¹⁾CRODAFOS CS20 Acid is Ceteth-20 & Cetaryl Alcohol & Dicetyl Phosphate; and
²⁾NATROSOL 250 HHR CS is hydroxyethylcellulose.

Samples A-C (1.8 ml) were applied to the thumb, index, and middle fingers of clean hands. Skin pH readings were taken prior to treatment (baseline), immediately after the fingers were dry, and again after two hours for Samples A and B and after four hours for Sample C. The averages of the skin pH values are provided in the above table.

Sample A containing polyacrylic acid lowered the skin pH to the greatest extent with a final skin pH after two hours of pH 4.7. Neither Sample B nor Sample C lowered the skin pH below pH 5.0. This data indicates that polyacrylic acid has an ability to suppress skin pH and maintain a low skin pH for a least two hours.

The viral efficacy of Samples A-C against Rhinovirus 39 was also tested. A viral load of about 10 pfu was spread over the thumb, index, and middle fingers of each treated hand and allowed to dry for 10 minutes. The fingers then were rinsed with viral recovery broth and samples were serially diluted and plated on H1-HeLa cells. Viral titers were measured using the plaque assay. For both Samples B and C, 100% of the hands were positive for rhinovirus, which indicates little efficacy of these compositions against rhinovirus. In contrast, Sample A demonstrated a viral efficacy because only 63% of the hands were found positive for rhinovirus.

### EXAMPLE 10

Example 8 demonstrated that a synergism exists between polyacrylic acid and ethanol, which results in suppression of skin pH and antiviral efficacy. The following compositions were prepared to examine the effectiveness of polycarboxylic acid blends and a single polycarboxylic acid composition, each in combination with polyacrylic acid and ethanol, on antiviral efficacy. A preferred antiviral composition contains the least amount of organic acid required to demonstrate a persistent antiviral efficacy.

The compositions were applied to the fingerpads of clean hands. After the indicated times, about 10³ to 10⁴ pfu of Rhinovirus 39 was applied to the hands and allowed to dry for 10 minutes. The virus was recovered by rinsing the hands with viral recovery broth. The samples then were diluted serially in viral recovery broth and plated on H1-HeLa cells. Viral titers were determined by plaque assay. The percentage of hands that were positive for rhinovirus is summarized below.

| | | | % of Hands |
|---|---|---|---|
| | | | Positive for |
| Composition (by wt %) | Time | | Rhinovirus |
| 70% ethanol | 15 | min. | 100% |
| 1% citric acid/1% malic acid/10% | 1 | hr. | 100% |
| ethanol/water | | | |
| 1% polyacrylic acid/4% citric acid/70% | 4 | hrs. | 91% |
| ethanol/water | | | |
| 1% polyacrylic acid/1% citric acid/1% | 4 | hrs. | 0% |
| malic acid/70% ethanol/water | | | |

A composition containing 70% ethanol alone was not effective as an antiviral composition. Citric acid (1%) and malic acid (1%) lost effectiveness against rhinovirus after one hour because 100% of the hands were found to be positive for rhinovirus. In contrast, when a composition containing 1% citric and 1% malic acids are applied to the hands in combination with polyacrylic acid and 70% ethanol, no virus was detected on the hands after four hours. A single acid (4% citric acid) in combination with a polyacrylic acid and ethanol was less effective against rhinovirus because 91% of hands were found to be positive for rhinovirus after four hours.

This data demonstrates that using a polyacrylic acid and ethanol allows the use of a lower concentration of polycarboxylic acid to achieve a desired antiviral efficacy. This improvement is attributed, at least in part, to forming a residual film or layer of the organic acids on the skin.

### EXAMPLE 11

The use of a polyacrylic acid and ethanol in a composition suppresses skin pH to a value below the solution pH, as demonstrated in Example 8. To test whether antiviral compositions containing citric acid, malic acid, polyacrylic acid, and ethanol can be buffered to a higher solution pH and still provide a skin pH at or below pH 4 to obtain a persistent antiviral activity, the following compositions were prepared.

| | | | Skin | Skin | |
|---|---|---|---|---|---|
| | | Solution | pH | pH | Viral |
| Sample | Composition (by wt %) | pH | Initial | 4 hrs. | Reduction |
| A | 1% ULTREZ 20/2% | 3.2 | 2.9 | 3.7 | >3 log₁₀ |
| | citric acid/2% malic | | | | |
| | acid/70% ethanol | | | | |
| B | 1% ULTREZ 20/2% | 4.34 | 3.4 | 3.7 | >3 log₁₀ |
| | citric acid/2% malic | | | | |
| | acid/70% ethanol | | | | |
| C | 1% ULTREZ 20/2% | 4.65 | 3.6 | 3.8 | >3 log₁₀ |
| | citric acid/2% malic | | | | |
| | acid/70% ethanol | | | | |

The compositions (1.8 mL) were applied to the thumb, index, and middle fingers of clean hands. Skin pH readings were measured prior to treatment (baseline), immediately after the fingers were dry, and again after four hours. The average of the skin pH values are plotted above.

Initial skin pH of skin treated with Samples A-C were suppressed to between pH 2.9 and 3.6, wherein the lower the solution pH, the lower the initial skin pH. However, after four hours, the skin pH for all three compositions was about pH 3.7. Consistent with previous examples, solution pH did not predict subsequent skin pH.

The viral efficacy of Samples A-C against Rhinovirus 39 also was tested. A viral load of about 10³ pfu was spread over the thumb, index, and middle fingers of each treated hand and allowed to dry for 10 minutes. The fingers then were rinsed with viral recovery broth and samples were diluted serially and plated on H1-HeLa cells. Viral titers were measured using the plaque assay. No virus was recovered from any of the hands indicating that all three Samples A-C have antiviral efficacy.

This data demonstrates than when citric acid and malic acid are utilized in a composition in combination with a polyacrylic acid and ethanol, the pH of the solution can be buffered to a higher, e.g., milder and safer, pH for application to the skin, while still retaining an ability to suppress skin pH and exhibit antiviral activity. This result also is attributed, at least in part, to the residual layer or film of organic acid that remains on the skin after evaporation of volatile composition ingredients.

The following tests demonstrate that an antiviral composition can provide an essentially continuous barrier layer of organic acid on a treated surface. In particular, the following tests show that an antiviral composition resists rinsing from a treated surface, e.g., at least 50% of the nonvolatile composition ingredients (including the organic acid) remains on a treated surface after three rinsings, as determined from NMR and IR spectra. In addition, an effective antiviral amount of the nonvolatile composition ingredients remains on a treated surface after 10 rinsings, also determined using NMR and IR spectra.

In the following tests, an aqueous composition containing, by weight, 2% malic acid, 2% citric acid, 1% polyacrylic acid, 62% ethanol, and 0.5% hydroxyethylcellulose as a gelling agent (Composition A) was compared to an aqueous composition, containing 2% malic acid, 2% citric acid, and 62% ethanol (Composition B). The compositions were applied to a glass surface to provide a film. From infrared (IR) and nuclear magnetic resonance (NMR) spectra of the film taken after each rinse, it was determined that Composition B was completely rinsed from the surface after one rinsing with water. Composition B therefore failed to exhibit water resistance and failed to provide a film or layer of nonvolatile composition ingredients on the surface.

In contrast, IR and NMR spectra showed that Composition A provided a rinse-resistant film or layer of composition ingredients on the treated surface. The amount of composition ingredients that remained on the treated surface was reduced over the first three rinsings, then resisted further removal from the treated surface in subsequent rinses. The IR and NMR spectra showed that detectable and effective amounts of the nonvolatile composition ingredients remained on the treated surface after 10 water rinses.

Another test was performed to measure the contact angle of water on a surface. “Contact angle” is a measure of the wetting ability of water on a surface. In this test, Compositions A and B were applied to a glass surface and allowed to dry. Contact angle then was measured for glass treated with Compositions A and B, both unrinsed and rinsed, using deionized water. The contact angle of bare, i.e., untreated, glass also was measured as a control. The following table summarizes the results of the contact angle test.

| | Composition A | Composition A | Composition B | Composition B | Bare |
|---|---|---|---|---|---|
| | Unrinsed | Rinsed | Unrinsed | Rinsed | Glass |
| Avg | 45.96 | 72.66 | 6.69 | 41.51 | 38.47 |
| Reading | | | | | |
| (degrees) | | | | | |
| Change in | | 26.7 | | 34.8 | |
| degrees | | | | | |
| % Change | | 58.1 | | 520.2 | |

The contact angle data shows that Composition A modifies the glass surface and provides a persistent barrier film or layer on the glass surface. The data also shows that Composition B is rinsed from the surface because the contact angle after rinsing of Composition B is essentially the same as that of bare glass.

Another test was performed to demonstrate metal ion uptake by a residual film of Composition A. In this test, films of Composition A were formed on glass, dried at least 4 hours, then exposed to solutions having a 0.5 M concentration of metal ions. Samples then were analyzed by SEM scan. The data in the following table shows that a film resulting from Composition A effectively binds several types of metal ions. It is theorized, but not relied upon, that this is a surface phenomenon because no mechanism for transporting metal ions into the film is known.

Composition A Films on Glass (Metal-Soaked & Deionized Water Rinsed)
(unless otherwise specified)

| Soaking Solution | EDS atomic % | EDS wt % |
|---|---|---|
| 0.56 wt % CaCl₂ in | 0.63% Ca | 1.71% Ca |
| formula on 316 | | |
| SS-No Rinse | | |
| 0.1 M Ca on 316 SS | 0.13% Ca | 0.21% Ca |
| 0.5 M Ca on 316 SS | 0.34% Ca | 0.54% Ca |
| 0.5 M Ca w/ more | 0.07% Ca | 0.12% Ca |
| rinsing on 316 SS | | |
| 0.5 M Cu on 316 SS | 0.65% Cu | 1.59% Cu |
| 0.5 M Fe on Al 6061 | 0.41% Fe | 1.14% Fe |
| 0.5 M Zn on Al 6061 | 0.24% Zn | 0.90% Zn |
| Metal Coupon anzlysis | | |
| DI water on 316 SS | 0% Ca, 0% Cu, 0% | 0% Ca, 0% Cu, 0% Zn |
| Fe compensated for in | | |
| above datum | Zn | |
| DI water on Al 6061 | 0.07% Ca, 0.08% Fe, | 0.18% Ca, 0.29% Fe, |
| | 0.03% Cu [from Al] | 0.11% Cu [from Al] |

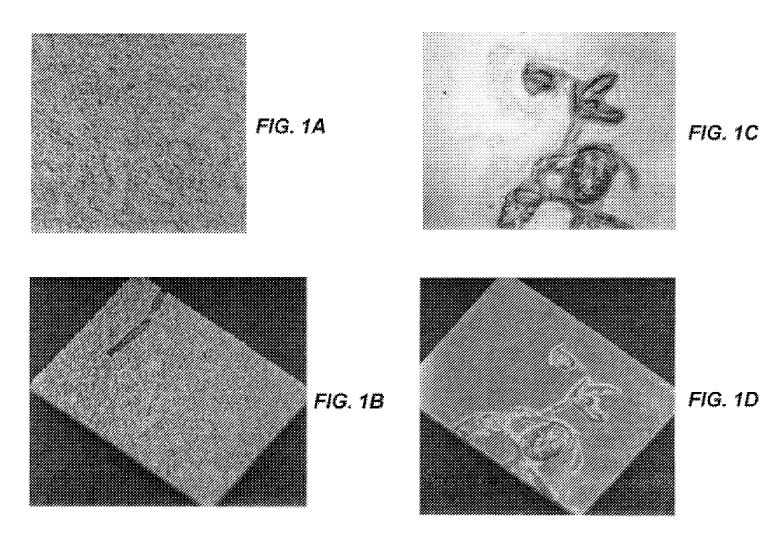

Reflectance micrographs showing the surface coverage of Compositions A and B also were taken (FIG. 1). The attached micrographs show that Composition A provides an essentially complete surface coverage, i.e., a more even coverage of Composition A on a treated surface, which provides an essentially continuous layer or film of nonvolatile composition ingredients on the surface. The attached micrographs are a digital conversion of reflectance values, which provide a direct correlation to surface coverage. The micrographs demonstrate that Composition A (FIGS. 1a) and 1b)) provides a film having improved adhesion, dispersion, and crystal formation compared to Composition B (FIGS. 1c) and 1d)).

### EXAMPLE 12

A time kill test was performed on additional bacteria and a fungus to demonstrate the broad spectrum efficacy of a composition of the present invention. In this test, the following antimicrobial composition was tested.

| Ingredient | Weight Percent |
|---|---|
| Cetyl Alcohol | 1.00 |
| Glycerin | 1.00 |
| Isopropyl Palmitate | 1.00 |
| Dimethicone 100 CST | 1.02 |
| Ethanol SDA-40B | 3.09 |
| Natrosol 250 HHX | 0.26 |
| Deionized Water | 10.94 |
| Deionized Water | 17.65 |
| ULTREZ 10 Polymer | 1.01 |
| Ethanol SDA-40B | 58.82 |
| Citric Acid | 2.00 |
| Malic Acid | 2.00 |
| Sodium Hydroxide 50% | 0.22 |

The above-composition was tested for an ability to control the following microorganisms under the following conditions:

| | |
|---|---|
| Test Systems: | Staphylococcus aureus ATCC 6538 |
| | Escherichia coli ATCC 11229 |
| | Listeria monocytogenes ATCC 7644 |
| | Enterobacter cloacae ATCC 13047 |
| | Candida albicans ATCC 10231 |
| Test Temperature: | Ambient (20-25° C.) |
| Exposure Time: | 15 and 30 seconds |
| Neutralizer | 99 mL of D/E Broth |
| | A neutralizer screen performed as part of the testing |
| | verified that the neutralizer adequately neutralized the |
| | products and was not detrimental to the |
| | tested organisms. |
| Subculture | D/E Agar |
| Medium: | |
| Incubation: | 35 ± 2° C. for 48 ± 4 hours (for S. aureus, E. coli, |
| | L. monocytogenes) |
| | 30 ± 2° C. for 48 ± 4 hours (for E. cloacae) |
| | 26 ± 2° C. for 72 ± 4 hours (for C. albicans) |

The test data summarized are below:

Inoculum Numbers (CFU/mL)

| Test System | A | B | Average |
|---|---|---|---|
| Staphylococcus aureus ATCC 6538 | 30 × 10⁶ | 29 × 10⁶ | 3.0 × 10⁷ |
| Escherichia coli ATCC 11229 | 18 × 10⁶ | 18 × 10⁶ | 1.8 × 10⁷ |
| Listeria monocytogenes ATCC 13047 | 26 × 10⁶ | 29 × 10⁶ | 2.8 × 10⁷ |
| Enterobacter cloacae ATCC 13047 | 31 × 10⁶ | 35 × 10⁶ | 3.3 × 10⁷ |
| Candida albicans ATCC 10231 | 24 × 10⁵ | 26 × 10⁵ | 2.5 × 10⁶ |

Staphylococcus aureus ATCC 15442

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | <100, <100 | <100 | >5.48 | >99.999 |
| 30 | <100, <100 | <100 | >5.48 | >99.999 |

Escherichia coli ATCC 11229

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | 2 × 10², <100 | <1.5 × 10² | >5.08 | >99.999 |
| 30 | <100, <100 | <100 | >5.26 | >99.999 |

Listeria monocytogenes ATCC 7644

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | <100, 3 × 10² | <2.0 × 10² | >5.15 | >99.999 |
| 30 | <100, <100 | <100 | >5.45 | >99.999 |

Enterobacter cloacae ATCC 13027

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, contamination | <100 | >5.52 | >99.999 |
| 30 | 5 × 10², 6 × 10² | 5.5 × 10² | 4.78 | >99.998 |

Candida albicans ATCC 10231

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | <100, <100 | <100 | >4.40 | >99.996 |
| 30 | <100, <100 | <100 | >4.40 | >99.996 |

The data shows that a composition of present invention exhibits about g reduction at 15 and 30 seconds of exposure time against Staphylococcus aureus ATCC 6538, Escherichia coli ATCC 11229, Listeria monocytogenes ATCC 7644, Enterobacter cloacae ATCC 13047, and Candida albicans ATCC 10231.

The above data shows that a present antimicrobial composition also is effective in controlling fungi, including yeasts and molds. Fungi control is important because fungi can cause a number of plant and animal diseases. For example, in humans, fungi cause ringworm, athlete's foot, and several additional serious diseases. Because fungi are more chemically and genetically similar to animals than other organisms, fungal diseases are very difficult to treat. Accordingly, prevention of fungal disease is desired. The prototype activity against fungi was examined using the yeast Candida albicans. The genus Candida contains a number of species, however, Candida albicans was tested because it is the most frequent cause of candidiasis. Candida albicans can be found in the alimentary tract, mouth, and vaginal area, and can cause diseases including oral candidiasis, also called thrush, vaginitis, alimentary candidiasis, and cutaneous and systemic candidiasis. In particular, a present invention is efficacious in controlling yeasts, such as Candida albicans, demonstrating a log reduction of at least 4 after a 15 second exposure time to a present antimicrobial composition.

The antiviral compositions used in the present method, independently, can be liquids, gels, semisolids, emulsions, lotions, creams, pastes, ointments, and the like. A liquid product can be a solution, dispersion, emulsion, or a similar product form. Gel and semisolid product forms can be transparent or opaque, designed for application by stick dispenser or by the fingers, or example. The preconditioning compositions typically are liquids or emulsions, but can be a bar soap. Additional types of compositions include foamed compositions, such as creams, mousses, and the like, and compositions containing organic and inorganic filler materials also can be used. The preconditioning and antiviral compositions can be manufactured as dilute ready-to-use compositions, or as concentrates that are diluted prior to use.

The preconditioning and antiviral compositions also can be incorporated, independently, into a web material or swab to provide wiping articles. The wiping articles can be used sequentially on animate surfaces by wiping in accordance with the present method.

In one embodiment of the present invention, an individual who either is suffering from a virus infection, or is likely to be exposed to other individuals suffering from a virus infection, such as a rhinovirus or a rotaviral infection, can apply a preconditioning composition, then an antiviral composition to his or her skin. This method of application improves inactivation of rhinovirus, and other nonenveloped virus particles, present on the skin. The method also can inactivate influenza viruses, noroviruses, and rotaviruses. The compositions as applied in the present method, either rinsed off or allowed to remain on the skin, provide a persistent antiviral activity. Nonenveloped viruses, like rhinovirus, or an influenza virus, therefore, are not transmitted to noninfected individuals via hand-to-hand transmission. The frequency of application, and the period of use will vary depending upon the level of disinfection desired, e.g., the degree of microbial contamination and/or skin soiling.

The present method provides the advantages of a persistent antiviral activity to the treated animate surface, and a broad spectrum viral control, in short contact times. The short contact time for a substantial log reduction of viruses is important in view of the typical 15 to 60 second time frame used to sanitize the skin. The antiviral compositions are effective in a short contact time because (a) the preconditioning step removes soil and sebum that inhibits contact of the antiviral composition with the skin and also standardizes, and preferentially lowers, skin pH, (b) the reduced pH of the composition, (c) the synergistic effect provided by the combination of a disinfecting alcohol and an organic acid, and (d) a persistent activity is enhanced because of a residual barrier layer or film of antiviral composition ingredients that can remain on the skin after evaporation of the volatile components of the composition.

Obviously, many modifications and variations of the invention as hereinbefore set forth can be made without departing from the spirit and scope thereof, and, therefore, only such limitations should be imposed as are indicated by the appended claims.

What is claimed is:

1. A method of reducing a virus population on an animate surface comprising: (a) contacting the surface with preconditioning composition, then (b) contacting the surface with an antiviral composition capable of achieving a log reduction of at least 4 against a nonenveloped virus after 30 seconds of contact.

2. The method of claim 1 wherein the preconditioning composition is a skin cleansing composition comprising an anionic surfactant, a nonionic surfactant, an amphoteric surfactant, or a mixture thereof.

3. The method of claim 1 wherein the preconditioning composition has a pH of about 5 to about 7.

4. The method of claim 2 wherein the preconditioning composition further comprises an antibacterial agent and at least one of a hydric solvent and a hydrotrope.

5. The method of claim 4 wherein the preconditioning composition comprises about 0.01% to about 2%, by weight, of the an antibacterial agent.

6. The method of claim 4 wherein the antibacterial agent comprises a phenolic antibacterial agent selected from the group consisting of:
(a) a 2-hydroxydiphenyl compound having the structure 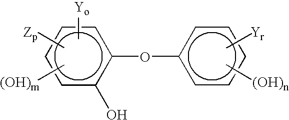
wherein Y is chlorine or bromine, Z is SO₃H, NO₂, or C₁-C₄ alkyl, r is to 3, o is 0 to 3, p is 0 or 1, m is 0 or 1, and n is 0 or 1;
(b) a phenol derivative having the structure 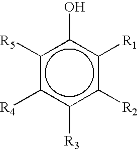
wherein R₁ is hydro, hydroxy, C₁-C₄ alkyl, chloro, nitro, phenyl, or benzyl, R₂ is hydro, hydroxy, C₁-C₆ alkyl, or halo, R₃ is hydro, C₁-C₆ alkyl, hydroxy, chloro, nitro, or a sulfur in the form of an alkali metal salt or ammonium salt, R₄ is hydro or methyl, and R₅ is hydro or nitro;
(c) a diphenyl compound having the structure 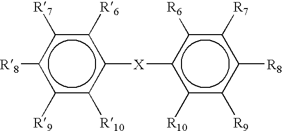
wherein X is sulfur or a methylene group, R₆ and R₁₆ are hydroxy, and
R₇, R′₇, R₈, R′₈, R₉, R′₉, R₁₀, and R′₁₀, independent of one another, are hydro or halo; and
(d) mixtures thereof.

7. The method of claim 1 wherein the preconditioning composition is rinsed from the skin prior to application of the antiviral composition.

8. The method of claim 1 wherein the preconditioning composition is allowed to remain on skin.

9. The method of claim 1 wherein the preconditioning composition reduces skin pH.

10. The method of claim 1 wherein the animate surface is a skin of a mammal.

11. The method of claim 1 wherein the nonenveloped virus is an acid-labile virus.

12. The method of claim 11 wherein the acid-labile virus comprises a rhinovirus serotype.

13. The method of claim 1 wherein the antiviral composition lowers a pH of the animate surface to less than 4.

14. The method of claim 1 wherein the antiviral composition is rinsed from the skin.

15. The method of claim 1 wherein the antiviral composition is allowed to remain on the surface and dry.

16. The method of claim 1 wherein the animate surface has a persistent antiviral activity.

17. The method of claim 1 wherein the antiviral composition comprises:
(a) about 25% to about 75%, by weight, of a disinfecting alcohol;
(b) a virucidally effective amount of an organic acid; and
(c) 0% to about 5%, by weight, of a gelling agent, and has a pH of about 2 to about 5.

18. The method of claim 1 wherein the antiviral composition forms a barrier layer comprising the organic acid on the animate surface.

19. The method of claim 1 wherein an essentially continuous layer comprising the organic acid is formed on the animate surface.

20. The method of claim 17 wherein the disinfecting alcohol is a C₁₋₆ alcohol or mixtures thereof.

21. The method of claim 20 wherein the disinfecting alcohol is selected from the group consisting of methanol, ethanol, isopropyl alcohol, n-butanol, n-propyl alcohol, and mixtures thereof.

22. The method of claim 17 wherein the composition comprises about 0.05% to about 15%, by weight, of the organic acid.

23. The method of claim 17 wherein the organic acid in the composition has a log P of less than one.

24. The method of claim 17 wherein the organic acid in the composition has a log P of one or greater.

25. The method of claim 17 wherein the organic acid comprises a first organic acid having a log P of less than one and a second organic acid having a log P of one or greater.

26. The method of claim 17 wherein the organic acid has a water solubility of at least about 0.05% by weight, at 25° C.

27. The method of claim 17 wherein the gelling agent is present in the antiviral composition in an amount of about 0.1% to about 3%, by weight.

28. The method of claim 17 wherein the gelling agent comprises a natural gum, a synthetic polymer, a clay, an oil, a wax, and mixtures thereof.

29. The method of claim 17 wherein the gelling agent is selected from the group consisting of cellulose, a cellulose derivative, guar, a guar derivative, algin, an algin derivative, a water-insoluble C₈-C₂₀ alcohol, carrageenan, a smectite clay, a polyquatemium compound, and mixtures thereof.

30. The method of claim 1 wherein the antiviral composition is free of a surfactant.

31. The method of claim 17 wherein the antiviral composition further comprises a hydrotrope in amount of about 0.1% to about 30%, by weight.

32. The method of claim 17 wherein the antiviral composition further comprises about 0.1% to about 30% of a polyhydric solvent selected from the group consisting of a diol, a triol, and mixtures thereof.

33. The method of claim 18 wherein a virucidally effective amount of the organic acid remains in the barrier layer on the animate surface after ten rinsings with water.

34. The method of claim 18 wherein at least 50%, by weight, of the nonvolatile components of the antiviral composition are present on the animate surface after three rinses with water.

35. The method of claim 17 wherein the organic acid comprises one or more of a monocarboxylic acid, a polycarboxylic acid, a polymeric acid having a plurality of carboxylic, phosphate, sulfonate, and/or sulfate moieties, anhydrides thereof, or mixtures thereof.

36. The method of claim 35 wherein the organic acid comprises a monocarboxylic acid having a structure RCO₂H, wherein R is C₁₋₃alkyl, hydroxyC₁₋₃alkyl, haloC₁₋₃alkyl, phenyl, or substituted phenyl.

37. The method of claim 35 wherein the monocarboxylic acid is selected from the group consisting of acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof.

38. The method of claim 17 wherein organic acid comprises a polycarboxylic acid containing two to four carboxylic acid groups, and optionally contains one or more hydroxyl group, amino group, or both.

39. The method of claim 38 wherein the polycarboxylic acid is selected from the group consisting of malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

40. The method of claim 17 wherein the organic acid comprises a polymeric acid having a molecular weight of about 500 to about 10,000,000 g/mol.

41. The method of claim 40 wherein the polymeric acid is water soluble or water dispersible.

42. The method of claim 40 wherein the polymeric acid is selected from the group consisting of a polymeric carboxylic acid, a polymeric sulfonic acid, a sulfated polymer, a polymeric phosphoric acid, and mixtures thereof.

43. The method of claim 40 wherein the polymeric acid comprises a homopolymer or a copolymer of acrylic acid.

44. The method of claim 17 wherein the organic acid comprises a polycarboxylic acid and a polymeric carboxylic acid.

45. The method of claim 44 wherein the polymeric acid comprises a homopolymer or a copolymer of acrylic acid.

46. The method of claim 44 wherein the polycarboxylic acid comprises citric acid, malic acid, tartaric acid, and mixtures thereof, and the polymeric carboxylic acid comprises a homopolymer or a copolymer of acrylic acid or methacrylic acid.

47. The method of claim 17 wherein the antiviral composition has a pH of about 2 to about 4.

48. The method of claim 1 wherein the animate surface has a pH or less than 4 four hours after contact with the antiviral composition.

49. The method of claim 1 wherein a log reduction of at least 3 against a nonenveloped virus is acheived at least about four hours after contact with the antiviral agent.

50. The method of claim 1 wherein a log reduction of at least 2 against a nonenveloped virus is acheived about six hours after contact with the antiviral agent.

51. The method of claim 1 wherein the virus is an influenza virus, a rotavirus, or a norovirus.

52. The method of claim 1 wherein the composition further reduces a fungus population on the animate surface.

53. The method of claim 52 wherein the fungus comprises a mold, a yeast, or both.

54. The method of claim 53 wherein the fungus comprises a yeast.

55. The method of claim 54 wherein the yeast comprises Candida albicans.

56. The method of claim 52 wherein the composition imparts a log reduction of at least 4 against Candida albicans on the animate surface after a 15 second exposure to the composition.

57. A method of inactivating viruses on an animate surface comprising (a) contacting the surface with a preconditioning composition, then (b) contacting the surface with an antiviral composition capable of achieving a log reduction of at least 4 against a nonenveloped virus after 30 seconds of contact.

58. The method of claim 57 wherein a persistent antiviral efficacy is imparted to the surface.

59. The method of claim 57 wherein nonenveloped viruses are inactivated.

60. The method of claim 57 wherein influenza viruses and rotaviruses are inactivated.

61. The method of claim 57 wherein rhinoviruses, picornaviruses, adenoviruses, herpes viruses, respiratory syncytial viruses, coronaviruses, enteroviruses, and similar pathogenic viruses are inactivated.

62. The method of claim 57 wherein acid-labile viruses are inactivated.

63. The method of claim 57 wherein picornaviruses are inactivated.

64. The method of claim 57 wherein rhinoviruses are inactivated.

65. A method of protecting an individual against infection by a virus comprising (a) contacting skin of the individual with a preconditioning composition, then (b) contacting the skin with an antiviral composition capable of achieving a log reduction of at least 4 against a nonenveloped virus after 30 seconds of contact.

66. The method of claim 65 wherein the preconditioning composition and antiviral composition are applied prior to the individual being exposed to the virus.

67. The method of claim 65 wherein the preconditioning composition and the antiviral composition are applied multiple times within a twenty-four-hour period.

68. A kit capable of providing a persistent control of viruses on an animate surface, said kit comprising:
(a) a first package comprising a preconditioning composition for application to the animate surface;
(b) a second package comprising an antiviral composition for application to the animate surface; and
(c) indicia comprising instructions to apply the preconditioning composition to the animate surface prior to the antiviral composition.

69. The kit of claim 68 wherein the indicia further comprises instructions to allow the antiviral composition to remain on the animate surface after application.

70. The kit of claim 68 wherein the indicia further comprises instructions to rinse the preconditioning composition from the animate surface prior to applying the antiviral composition.

71. The kit of claim 68 further comprising a first dispenser for the preconditioning composition and a second dispenser for the antiviral composition.

72. The kit of claim 71 wherein the first dispenser dispenses a measured effective dose of the preconditioning composition and the second dispenser dispenses a measured effective dose of the antiviral composition.
